(19) United States
(12) Patent Application Publication
(10) Pub. No.: US 2023/0225665 A1
(43) Pub. Date: Jul. 20, 2023

(54) SYSTEMS AND METHODS FOR DETECTION OF DELIRIUM AND OTHER NEUROLOGICAL CONDITIONS

(71) Applicant: Ceribell, Inc., Sunnyvale, CA (US)

(72) Inventors: Baharan KAMOUSI, Redwood City, CA (US); Suganya Karunakaran, Sunnyvale, CA (US); Archit Gupta, Sunnyvale, CA (US); Raymond Woo, Los Altos, CA (US); Xingjuan Chao, Palo Alto, CA (US)

(21) Appl. No.: 18/153,986

(22) Filed: Jan. 12, 2023

Related U.S. Application Data
(60) Provisional application No. 63/298,937, filed on Jan. 12, 2022.

(51) Int. Cl. A61B 5/372; G16H 10/60; G16H 50/20; A61B 5/31; A61B 5/384; A61B 5/00

(52) U.S. Cl. CPC A61B 5/372; G16H 10/60; G16H 50/20; A61B 5/31; A61B 5/384; A61B 5/6803

(57) ABSTRACT

Described herein are systems and methods for the detection and monitoring of delirium in a subject. Other neurological conditions may also be detected and monitored. The systems may include a data module configured to obtain a plurality of electroencephalography (EEG) signals collected from a subject. The systems may also include a processing module in communication with the data module. The processing module may be configured to process the data to detect and monitor delirium and/or one or more other neurological conditions that the subject is experiencing or likely to experience. The processing module may also generate indications or assessments for delirium and/or for each neurological condition at an individual level, or optionally, between two or more related neurological conditions.

### CROSS-REFERENCE TO RELATED APPLICATIONS

This application claims priority to U.S. Provisional Application No. 63/298,937, filed on Jan. 12, 2022, which is hereby incorporated by reference in its entirety.

### FIELD

This application relates to systems and methods for the detection and monitoring of delirium in a subject. The detection and monitoring of delirium may be based on output from one or more machine learning models that process a plurality of features extracted from electroencephalography (EEG) signals. The output of the one or more machine learning models may comprise, or be used to generate, a delirium trend of the subject over a period of time. The systems and methods may also be employed to detect and monitor other neurological conditions or brain function abnormalities such as seizure, stroke, and sedation, as well as differentiate between the conditions.

### BACKGROUND

Delirium is a clinical state that manifests as an acute disturbance in cognition, and is common amongst hospitalized patients and particularly common with certain high risk patient populations. The American Psychiatric Association's Diagnostic and Statistical Manual of Mental Disorders, 5^{th} edition (DSM-V) details the following diagnostic criteria for delirium: a) a disturbance in attention (i.e., a reduced ability to direct, focus, sustain, and shift attention) and awareness (reduced orientation to the environment); b) a disturbance that develops over a short period of time (usually hours to a few days), and which is an acute change from baseline attention and awareness, and tends to fluctuate in severity during the course of a day; c) an additional disturbance in cognition (e.g., memory deficit, disorientation, language, visuospatial ability, or perception); d) the disturbances in criteria a) and b) are not better explained by a pre-existing, established, or evolving neurocognitive disorder; and e) there is evidence from the history, physical examination, or laboratory findings that the disturbance is a direct physiological consequence of another medical condition, substance intoxication, or withdrawal (i.e., due to a drug of abuse or to a medication), or exposure to a toxin, or is due to multiple etiologies.

Delirium may present in a hypoactive or hyperactive state. Hyperactive delirium is characterized by an increase in activity and can include restless, agitated, or aggressive behavior. Hypoactive delirium is characterized by a decrease in activity and can include lethargy, abnormal drowsiness, or withdrawal. Mixed delirium refers to a fluctuation between hypoactive and hyperactive delirium states.

Failure to diagnose delirium has been shown to significantly impact patient mortality. Monitoring of delirium and other neurological conditions, e.g., sedation, has been traditionally performed via clinical assessment using a variety of assessment scales by a trained health professional, doctor, or nurse. This practice may be problematic as the assessment methods generally rely on physical patient movement or reaction, making subtle discrimination of various sedation and delirium levels difficult. For example, the current standard of care (SOC) for delirium assessment in acute care settings is the CAM-ICU (Confusion Assessment Matrix—ICU). Although the CAM-ICU was designed specifically for use in intensive care units, it has become the SOC for all critical care situations including emergency departments and other critical care settings. The CAM-ICU assessment is typically performed by the bedside nurse, and it consists of a series of assessment features, as follows:
Feature 1: Acute Onset or Fluctuating Course.
   Is the patient different than his/her baseline mental status OR has the patient had any fluctuation in mental status in the past 24 hours as evidenced by fluctuation on a sedation/level of consciousness scale?
Feature 2: Inattention.
   The assessor slowly reads a series of 10 letters. The patient is asked to squeeze the assessor's hand every time they hear the letter ‘A.’ This feature is failed if the patient makes more than 2 errors.
Feature 3: Altered Level of Consciousness.
   Is the patient anything other than alert and calm? Using the Richmond Agitation and Sedation Scale (RASS) is the score anything other than 0?
Feature 4: Disorganized Thinking.
   The assessor asks a series of logic questions. This feature is failed if the patient makes more than 1 error:
      Will a stone float on water?
      Are there fish in the sea?
      Does one pound weigh more than two pounds?
      Can you use a hammer to pound a nail?

The patient is considered delirium positive if Feature 1, Feature 2, and either Feature 3 or 4 are present. The CAM-ICU assessment has been validated to be very effective when performed in research settings. However, it has become apparent that in real-world routine use, the effectiveness of delirium assessment tools is significantly worse compared to results obtained in research settings.

The most comprehensive study of the effectiveness of the CAM-ICU assessment in real-world routine use conditions involved 282 patients across 10 different hospital ICUs. In this study, the bedside clinical ICU nurses (who had all received training in the CAM-ICU), achieved a sensitivity of only 47% with a specificity of 98%. Over half of the patients with delirium were not detected using the standard-of-care clinical nurse assessments in the 10 participating hospitals. Other smaller studies have shown that in real-world routine settings, nurses can fail to recognize delirium 75% of the time.

The SOC has also not been found to assess delirium with sufficient frequency to adequately manage the fluctuating nature of delirium. The delirium assessment tools used in the current SOC (including the CAM-ICU), were designed to be performed once per shift by the bedside clinical nurse. This means that at a maximum, delirium assessment is performed twice per day (once every 12 hours). At some hospitals, delirium assessment is only performed once per day, during the morning round by a medical team. As a result, SOC delirium assessments may experience a 12 to 24 hour delay in the recognition of delirium. Studies have shown that the delayed treatment of delirium may result in a significant increase in patient morbidity and mortality.

Delirium is known to be multi-factorial with many possible causes and risk factors. A key first step in the management of a patient with delirium is to recognize the risk factors and reduce or eliminate the risk factors as much as possible. Some of the delirium risk factors, such as age, are not modifiable. But other risk factors are potentially modifiable, such as the use of delirium causing medication. Treating delirium may consist of non-pharmacological interventions as well as pharmacological interventions. Non-pharmacological interventions include sleep optimization, re-orientation, increased movement, family involvement, and other sensory/behavior interventions. Pharmacological intervention can include stopping or changing existing medications that increase risk of delirium, or administering medication intended to reduce or mitigate delirium.

Given the multi-factorial nature of delirium and the many possible treatment options, monitoring treatment effectiveness may be a key part of optimizing treatment and minimizing delirium duration. However, the SOC delirium assessment methods are not able to provide ongoing monitoring due to the low frequency nature of the assessments. Accordingly, it would be beneficial to have alternative methods and systems for detecting and monitoring delirium. It would also be helpful to have new methods and systems for detecting and monitoring other neurological conditions.

### SUMMARY

Described herein are systems and methods that may detect and monitor delirium in a subject rapidly and accurately using EEG signals and machine learning. The detection and monitoring of delirium may be based on output from one or more machine learning models that process a plurality of features extracted from the EEG signals. The output of the one or more machine learning models may comprise, or be used to generate, a delirium trend of the subject over a period of time. If delirium is detected, any suitable therapy may be given to the subject to treat the delirium (e.g., a drug, controlling the environment, addressing an underlying medical condition). The systems and methods may also be employed to detect and monitor other neurological conditions or brain function abnormalities such as seizure, stroke, and sedation, as well as differentiate between the conditions.

In general, the systems and methods include a machine learning model that may be trained to use one or more features of EEG signals and output a delirium-positive or delirium-negative assessment. The EEG features that contribute to the machine learning model may include both time-domain and frequency-domain characteristics.

Methods for detecting and/or monitoring delirium are described herein. In one aspect, the method for detecting delirium includes obtaining data comprising a plurality of electroencephalography (EEG) signals recorded over one or more channels or a plurality of channels from a subject, and pre-processing the data by dividing the EEG signal into a plurality of temporal segments, where each temporal segment corresponds to a time epoch defined by at least a start time and a duration. A plurality of features from each of the plurality of temporal segments may then be extracted and one or more machine learning models used to generate a delirium classification for each of the temporal segments based on the extracted plurality of features. Thereafter, an overall delirium score for the subject may then be determined during a time-window, where the overall delirium score may be based on the delirium classifications generated by the one or more machine learning models, and the time window that includes one or more time epochs. The number of channels over which the EEG signals are recorded may range from 1 to 45, including all values and sub-ranges therein. For example, the number of channels may include 1 channel, 2 channels, 3 channels, 4 channels, 5 channels, 6 channels, 7 channels, 8 channels, 9 channels, 10 channels, 11 channels, 12 channels, 13 channels, 14 channels, 15 channels, 16 channels, 17 channels, 18 channels, 19 channels, 20 channels, 21 channels, 22 channels, 23 channels, 24 channels, 25 channels, 26 channels, 27 channels, 28 channels, 29 channels, 30 channels, 31 channels, 32 channels, 33 channels, 34 channels, 35 channels, 36 channels, 37 channels, 38 channels, 39 channels, 40 channels, 41 channels, 42 channels, 43 channels, 44 channels, or 45 channels. The plurality of channels may comprise a plurality of electrodes, which may be coupled to or incorporated into a headband, headgear, or other apparatus configured to place the electrodes on or around the head of the patient.

Systems for detecting and/or monitoring delirium are also described herein. In one aspect, the system includes a data module configured to receive data comprising a plurality of electroencephalography (EEG) signals recorded during a time window and over one or more channels or a plurality of channels from a subject, and a delirium detection module comprising a memory storing a set of instructions, and one or more processors that are configured to, and in response to the set of instructions, pre-process the data received by the data module. Pre-processing may include dividing the EEG signals into a plurality of temporal segments, where each temporal segment corresponds to a time epoch defined by at least a start time and a duration, and extracting a plurality of features from each of the plurality of temporal segments. Furthermore, the one or more machine learning models may be used to generate a delirium classification for each of the temporal segments based on the extracted plurality of features. An overall delirium score may be determined based on the delirium classifications generated by the one or more machine learning models. The number of channels in the system may range from 1 to 45, including all values and sub-ranges therein. For example, the number of channels may include 1 channel, 2 channels, 3 channels, 4 channels, 5 channels, 6 channels, 7 channels, 8 channels, 9 channels, 10 channels, 11 channels, 12 channels, 13 channels, 14 channels, 15 channels, 16 channels, 17 channels, 18 channels, 19 channels, 20 channels, 21 channels, 22 channels, 23 channels, 24 channels, 25 channels, 26 channels, 27 channels, 28 channels, 29 channels, 30 channels, 31 channels, 32 channels, 33 channels, 34 channels, 35 channels, 36 channels, 37 channels, 38 channels, 39 channels, 40 channels, 41 channels, 42 channels, 43 channels, 44 channels, or 45 channels. The plurality of channels may comprise a plurality of electrodes, as described above, and thus the system may include a headband, headgear, or other apparatus configured to place the electrodes on or around the head of the patient.

Additionally, methods for detecting a brain function abnormality are disclosed herein. In one aspect, the method includes obtaining data comprising a plurality of electroencephalography (EEG) signals recorded during a time window and over a plurality of channels from a subject, and pre-processing the data. Pre-processing may include dividing the EEG signals into a plurality of time-based segments, where each time-based segment corresponds to a time epoch defined by at least a start time and a duration, and extracting a plurality of features from each of the plurality of time-based segments. One or more machine learning models may be used to generate a plurality of classifications for each of the time-based segments based on the extracted plurality of features, where the plurality of classifications comprise, for each time-based segment, a separate classification for each of two or more indications selected from the group consisting of sedation, delirium, stroke, or seizure. One or more measures of brain function abnormality (BFA) may be displayed that are responsive to the plurality of classifications.

Systems for detecting a BFA are further described herein. In one aspect, the system includes a data module configured to receive data comprising a plurality of electroencephalography (EEG) signals recorded during a time window and over a plurality of channels from a subject, and a BFA detection module comprising a memory storing a set of instructions and one or more processors that are configured to, in response to the set of instructions, pre-process the data. Pre-processing may include dividing the EEG signals into a plurality of time-based segments, where each time-based segment corresponds to a time epoch defined by at least a start time and a duration, and extracting a plurality of features from each of the plurality of time-based segments. One or more machine learning models may be used to generate a plurality of classifications for each of the time-based segments based on the extracted plurality of features, where the plurality of classifications comprise, for each time-based segment, a separate classification for each of two or more indications selected from the group consisting of sedation, delirium, stroke, or seizure. One or more measures of BFA may be displayed that are responsive to the plurality of classifications.

In another aspect, the present disclosure provides a neurological condition detection and monitoring system. The system may include a data module configured to obtain data comprising a plurality of electroencephalography (EEG) signals collected from a subject. The system may also include a processing module in communication with the data module. The processing module may be configured to process the data to detect and monitor one or more neurological conditions that the subject is experiencing or likely to experience. The processing module may be also configured to generate indications or assessments (i) for each neurological condition at an individual level and optionally (ii) between two or more related neurological conditions. In some cases, the one or more neurological conditions relate to at least one of sedation, delirium, stroke, or seizure.

The processing module may be configured to process the data to simultaneously detect and monitor the one or more neurological conditions in real-time.

The two or more related neurological conditions may include sedation and delirium. The indications or assessments generated by the processing module may be indicative of a relationship or degree of correlation between sedation and delirium.

The data module may include a plurality of electrodes that are configured to be placed on different regions of the subject's head, wherein the different regions comprise frontal lobes, temporal lobes, and occipital lobes. The data module further may also include a plurality of channels that multiplexes the EEG signals from the plurality of electrodes in each region and between the different regions. The data further may also include non-EEG data. The non-EEG data may include one or more of blood pressure, heart rate, or motion data of the subject.

The processing module may be further configured to convert the data into one or more corollary assessment scores that are based at least on Riker Sedation-Agitation Scale (SAS), Richmond Agitation and Sedation Scale (RASS), Confusion Assessment Method Intensive Care Unit (CAM-ICU), CAM-ICU-7, the Delirium Rating Scale Revised (DRS-R-98), the Intensive Care Delirium Screening Checklist (ICDSC), or one or more applicable scales for one or more indications.

The processing module may be further configured to generate a visual output comprising a graph that displays a probability that the subject is experiencing delirium and/or a severity of the delirium, on a probability/severity scale as a function of time. The processing module may be further configured to generate one or more corollary assessment scores that are indicative of the severity of delirium. The processing module may be further configured to generate a diagnostic output based on the indications or assessments. The diagnostic output may include an aggregate wellness score or a graphical representation of the subject's brain state. The aggregate wellness score may be a combination of a plurality of discrete scores corresponding to the plurality of neurological conditions. The plurality of discrete scores may be combined based on different weights allocated to the plurality of neurological conditions. The graphical representation may be a combination of a plurality of different temporal graphs corresponding to the plurality of neurological conditions. The graphical representation comprises an overlay of the plurality of different temporal graphs.

The processing module may be configured to process the data to detect and analyze a plurality of features that are likely to be associated with the plurality of neurological conditions. The plurality of features may include a plurality of time-domain features and frequency-domain features. The plurality of features may include brain asymmetry, amplitude variations, spatial and temporal correlations, coherences, or co-variations of two or more features. The plurality of features may be further ranked and classified.

The processing module is further configured to use the plurality of features as inputs to train a machine learning algorithm for classifying different classes or severity relating to the one or more neurological conditions.

In another aspect, the present disclosure provides a neurological condition detection and monitoring method. The method may include obtaining data comprising a plurality of electroencephalography (EEG) signals collected from a subject. The method may also include processing the data to (1) detect and monitor one or more neurological conditions that the subject is experiencing or likely to experience, and (2) generate indications or assessments (i) for each neurological condition at an individual level and optionally (ii) between two or more related neurological conditions. The one or more neurological conditions is selected from the group consisting of sedation, delirium, stroke, and seizure.

### BRIEF DESCRIPTION OF THE DRAWINGS

FIG. 1 illustrates a schematic of a neurological condition detection and monitoring system, according to embodiments herein.

FIG. 2 illustrates a schematic of the various modules for a neurological condition detection and monitoring system, according to embodiments herein.

FIG. 3 illustrates a schematic of an interface of a neurological condition detection and monitoring system, according to embodiments herein.

FIG. 4 illustrates a time series plot of the neurological conditions for a patient, according to embodiments herein.

FIG. 5 illustrates various meters for various neurological conditions, according to embodiments herein.

FIG. 6 shows a schematic of a computer system that is programmed or otherwise configured to implement methods provided herein.

FIG. 7 shows a schematic of a delirium detection module in accordance with embodiments herein.

FIG. 8 shows a schematic of an alternative delirium detection module in accordance with embodiments herein.

FIG. 9 shows a schematic of another alternative delirium detection module in accordance with embodiments herein.

### DETAILED DESCRIPTION

This application relates to systems and methods for the detection and monitoring of delirium in a subject. The monitoring of delirium may be based on output from one or more machine learning models that process a plurality of features extracted from electroencephalography (EEG) signals. The output of the one or more machine learning models may comprise, or be used to generate, a delirium trend of the subject over a period of time. If delirium is detected, any suitable therapy may be given to the subject to treat the delirium (e.g., a drug, controlling the environment, addressing an underlying medical condition), as mentioned above. The systems and methods may also be employed to detect and monitor other neurological conditions or brain function abnormalities such as seizure, stroke, and sedation, as well as differentiate between the conditions.

### Delirium

Delirium may be an acute disturbance of consciousness and cognition that usually fluctuates over time, as previously stated. Delirium may be a common disorder, with reported incidences of more than 60% during Intensive Care Unit (ICU) stay and over 15% on a geriatric ward or medium care unit. Delirium may be associated with higher mortality, longer hospital stays, long-term cognitive impairment and increased costs. Delirium is typically divided into three different subtypes based on psychomotor behavior: hypoactive, hyperactive and mixed-type delirium. Hyperactive delirium may be characterized by increased motor activity, which may manifest as restlessness, agitation, aggression, wandering, and inappropriate behavior, as well as hyper-alertness, hallucinations and delusions. Hypoactive delirium may be characterized by reduced motor activity, which may manifest as lethargy, withdrawal, drowsiness and staring into space. Hypoactive delirium is the most common form of delirium in older people. Mixed-type delirium is characterized by a subject presenting aspects of both hypoactive and hyperactive delirium.

Despite the frequency and impact of delirium, recognition of delirium by health care professionals has been poor. Furthermore, delayed treatment of delirium in ICU patients has been found to increase mortality and morbidity. In order to improve early diagnosis and treatment, the Society of Critical Care Medicine and the American Psychiatric Association have recommended daily monitoring of delirium in ICU patients. Various delirium clinical assessment tools have been developed. Of these, the Confusion Assessment Method for the ICU (CAM-ICU) had highest sensitivity in ICU patients. However, the sensitivity of the CAM-ICU in routine, daily practice appeared to be low (overall 47%), particularly to detect the hypoactive type of delirium (sensitivity 31%) and delirium in postoperative patients. Unfortunately, the CAM-ICU has a limitation that it does not assess severity of delirium. Confusion Assessment Method (CAM)-ICU-7 is an alternative assessment that provides a delirium severity scale, which is 7-point scale (0-7), and it derived from responses to CAM-ICU and RASS items. However, the above-noted screening protocols generally do not fit well in the culture of the ICU that is typically orientated primarily on monitoring physiological changes in a patient, and using passive methods that do not depend on behavior-based assessments administered by a specialist.

These factors may hinder early treatment and may therefore impair outcome. Moreover, research on delirium in the ICU may be hampered by the lack of a sensitive tool for monitoring. Delirium may be accurately monitored using EEG and diagnosed using EEG-based biomarkers and machine learning algorithms described elsewhere herein.

In an aspect, the present disclosure provides a method for detecting delirium in a subject comprising: obtaining data comprising a plurality of electroencephalography (EEG) signals recorded over a plurality of channels from the subject; pre-processing the data by: dividing the EEG signal into a plurality of temporal segments, each temporal segment corresponding to a time epoch defined by at least a start time and a duration; and extracting a plurality of features from each of the plurality of temporal segments; using one or more machine learning models to generate a delirium classification for each of the temporal segments based on the extracted plurality of features; and determining an overall delirium score for the subject during a time-window, the overall delirium score being based on the delirium classifications generated by the one or more machine learning models, and the time window comprising one or more time epochs. In some embodiments, the delirium is hypo-active delirium.

In some embodiments, the subject may be selected based on having an increased risk for experiencing delirium. The increased delirium risk may be based on one or more of the following risk factors, which may be associated with ICU admission: benzodiazepine use, blood transfusion, age, dementia, prior delirium episodes, prior coma, emergency surgery, trauma, increasing Acute Physiology and Chronic Health Evaluation (APACHE) score and increasing American Society of Anesthesiologists (ASA) physical status classification system scores.

In some embodiments, the delirium classification is a binary classification that is delirium-positive or delirium-negative, a delirium probability value, or a delirium severity value. In some embodiments, the delirium classification may further classify delirium-positive cases into sub-types of hypoactive, hyperactive, or mixed type delirium. In some embodiments, the method further comprises providing a trace of the overall delirium score over time. The method may further comprise determining a trendline of the trace.

In some embodiments, the pre-processing of the data further comprises extracting a plurality of multi-channel features that quantify a degree of correlation between pairs of temporal segments from different EEG signals corresponding to a given time epoch; the method further comprises using a multichannel machine learning model to generate a multi-channel delirium classification for each time epoch based on the plurality of multi-channel features; and the delirium score is further based on the multi-channel delirium classification.

In some embodiments, the time-window has a duration that encompasses one time epoch, or a duration that encompasses a plurality of successive time epochs. In some embodiments, the duration of each of the time epochs is about 1 second and about 10 minutes. The duration of each of the time epochs may be about 10 seconds, about 30 seconds, about 60 seconds, about 2 minutes, about 5 minutes, or about 10 minutes. In some embodiments, successive time epochs may be non-overlapping, or may overlap by 50% or less.

In some embodiments, the plurality of features comprises at least one time-domain feature, at least one frequency-domain feature, or at least one feature that quantifies a degree of correlation of the time-based segment with a corresponding time-based segment of at least one other simultaneously collected EEG signal. The at least one other simultaneously collected EEG signal may be collected from a same hemisphere of the brain or from a different hemisphere of the brain.

In some embodiments, each channel is assigned to an independent machine learning model, and for each channel, the extracted features are applied to the machine learning model corresponding to the channel. In some embodiments, the one or more machine learning models is a random forest model. In some embodiments, the multichannel machine learning model is a random forest model.

In an aspect, the present disclosure provides a system for detecting delirium comprising: a data module configured to receive data comprising a plurality of electroencephalography (EEG) signals recorded during a time window and over a plurality of channels from a subject; and a delirium detection module comprising a memory storing a set of instructions and one or more processors that are configured to, responsive to the set of instructions: pre-process the data received by the data module by: dividing the EEG signal into a plurality of temporal segments, each temporal segment corresponding to a time epoch defined by at least a start time and a duration; and extracting a plurality of features from each of the plurality of temporal segments; use one or more machine learning models to generate a delirium classification for each of the temporal segments based on the extracted plurality of features; and determine an overall delirium score based on the delirium classifications generated by the one or more machine learning models. In certain embodiments, the delirium is hypo-active delirium.

In certain embodiments, the delirium classification is a binary score that is delirium-positive or delirium-negative. In certain embodiments, the overall delirium score of the subject is based on a percentage of the time-based segments within the time-window being delirium-positive, such that a higher percentage of delirium-positive time-based segments results in a higher delirium burden or delirium severity. In certain embodiments, the delirium classification comprises a delirium probability between 0 and 1. In certain embodiments, the delirium classification comprises a severity value of the degree of severity of the delirium.

In certain embodiments, the pre-processing of the data further comprises: extracting a plurality of multi-channel features that quantify a degree of correlation between pairs of time-based segments from different EEG signals corresponding to a given time epoch; and using a multichannel machine learning model to generate a multi-channel delirium classification for each time epoch based on the plurality of multi-channel features, wherein the overall delirium score is further based on the multi-channel delirium classification.

In certain embodiments, the time-window has a duration that encompasses one time epoch or a duration that encompasses a plurality of successive time epochs. In certain embodiments, the duration of the time epoch is between about 1 second and about 10 minutes. The duration of the time epoch may be about 10 seconds, about 30 seconds, about 60 seconds, about 2 minutes, about 5 minutes, or about 10 minutes. In certain embodiments, successive time epochs are non-overlapping, or overlap by 50% or less.

In certain embodiments, the plurality of features comprises at least one time-domain feature, at least one frequency-domain feature, or at least one feature that quantifies a degree of correlation of the time-based segment with a corresponding time-based segment of at least one other simultaneously collected EEG signal. The at least one other simultaneously collected EEG signal may be collected from a same hemisphere of the brain or from a different hemisphere of the brain.

In certain embodiments, each channel is assigned to an independent machine learning model, and wherein for each channel, the extracted features are applied to the machine learning model corresponding to the channel. In certain embodiments, the one or more machine learning models is a random forest model. In certain embodiments, the multichannel machine learning model is a random forest model.

In an aspect, the present disclosure provides a method for detecting a brain function abnormality (BFA) comprising: obtaining data comprising a plurality of electroencephalography (EEG) signals recorded during a time window and over a plurality of channels from a subject; pre-processing the data by: dividing the EEG signal into a plurality of time-based segments, each time-based segment corresponding to a time epoch defined by at least a start time and a duration; and extracting a plurality of features from each of the plurality of time-based segments; using one or more machine learning models to generate a plurality of classifications for each of the time-based segments based on the extracted plurality of features, wherein the plurality of classifications comprise, for each time-based segment, a separate classification for each of two or more indications selected from the group consisting of sedation, delirium, stroke, or seizure; and displaying one or more measures of BFA responsive to the plurality of classification.

In certain embodiments, the displaying of the one or more measures of BFA comprises displaying a separate measure of BFA corresponding to each of the two or more indications. In certain embodiments, the displaying of the one or more measures of BFA comprises displaying a combined measure of BFA based on the plurality of classifications that comprises, for each time-based segment, separate classifications for each of the two or more indications. In certain embodiments, the displaying of the one or more measures of BFA comprises selecting and displaying a most likely indication out of the two or more indications, based on the plurality of classifications that comprises, for each time-based segment, separate classifications for each of the two or more indications.

In certain embodiments, the separate classification for each of the two or more indications comprises a binary classification that is indication-positive or indication-negative. In certain embodiments, the separate classification for each of the two or more indications comprises a probability between 0 and 1 of the subject experiencing, within a given time-epoch, the two or more indications. In certain embodiments, the separate classification for each of the two or more indications comprises a severity value of the degree of severity of the two or more indications being experienced by the subject.

In certain embodiments, each channel is assigned to an independent machine learning model, and wherein for each channel, the extracted features are applied to the machine learning model corresponding to the channel. In certain embodiments, each of the two or more indications is assigned to an independent machine learning model, and wherein for each of the two or more indications, the extracted features are applied to the machine learning model corresponding to the indication.

In an aspect, the present disclosure provides a system for detecting a brain function abnormality (BFA) comprising: a data module configured to receive data comprising a plurality of electroencephalography (EEG) signals recorded during a time window and over a plurality of channels from a subject; and a BFA detection module comprising a memory storing a set of instructions and one or more processors that are configured to, in response to the set of instructions: pre-process the data by: dividing the EEG signal into a plurality of time-based segments, each time-based segment corresponding to a time epoch defined by at least a start time and a duration; and extracting a plurality of features from each of the plurality of time-based segments; use one or more machine learning models to generate a plurality of classifications for each of the time-based segments based on the extracted plurality of features, wherein the plurality of classifications comprise, for each time-based segment, a separate classification for each of two or more indications selected from the group consisting of sedation, delirium, stroke, or seizure; and display one or more measures of BFA responsive to the plurality of classifications.

In certain embodiments, the displaying of the one or more measures of BFA comprises displaying a separate measure of BFA corresponding to each the two or more indication. In certain embodiments, the displaying of the one or more measures of BFA comprises displaying a combined measure of BFA based on the plurality of classifications that comprises, for each time-based segment, separate classifications for each of the two or more indications. In certain embodiments, the displaying of the one or more measures of BFA comprises selecting and displaying a most likely indication out of the two or more indications, based on the plurality of classifications that comprises, for each time-based segment, separate classifications for each of the two or more indications.

In certain embodiments, the separate classification for each of the two or more indications comprises a binary classification that is indication-positive or indication-negative. In certain embodiments, the separate classification for each of the two or more indications comprises a probability between 0 and 1 of the subject experiencing, within a given time-epoch, the two or more indications. In certain embodiments, the separate classification comprises a severity value of the degree of severity of the two or more indications being experienced by the subject. In certain embodiments, each channel is assigned to an independent machine learning model, and wherein for each channel, the extracted features are applied to the machine learning model corresponding to the channel. In certain embodiments, each of the two or more indications is assigned to an independent machine learning model, and wherein for each of the two or more indications, the extracted features are applied to the machine learning model corresponding to the indication.

### Sedation

Sedation monitoring has been traditionally performed via clinical assessment by a trained health professional, doctor or nurse, using assessment scales like the Sedation-Agitation Scales (SAS), the Richmond Agitation Sedation Scale (RASS), or some similar variant, like the Ramsey Sedation Scale. These scales may categorize and translate certain clinical assessments, particularly in subjective assessments, into a numerical scale often ranging from a negative number (very sedated) to positive number (very awake). For instance, RASS ranges from −5 (Unarousable) to +4 (Combative). Using subjective clinical assessment scales to assess deeper levels of sedation may be problematic, as the assessment methods rely on physical patient movement or reaction and make subtle discrimination of deeper sedation levels difficult. Further, use of paralytics combined with sedatives, like during the use of a ventilator, may result in more challenging clinical assessments and can cause physician uncertainty as to whether the patient is appropriately sedated.

Objective, EEG-based sedation monitoring has been clinically adopted for use on surgical patients in the Operating Room (OR) by trained anesthesiologists. Anesthesiologists may be able to monitor various levels of sedation across various drug agents with more certainty and ease than clinical assessments during surgeries in concert with the other monitoring equipment traditionally used for sedation monitoring in the OR. There is a need for improved EEG-based sedation monitoring for widespread use outside of the OR in hospital units like the Post-Anesthesia Recovery Unit (PACU) and the Intensive Care Unit (ICU). In such hospital units, sedatives may be used for a range of reasons and procedures; short-term procedures like bronchoscopies or gastrointestinal procedures, continuous sedation for surgical patients requiring immobilization, or ventilated patients, some requiring additional paralytic agents. Among such situations in the ICU, continuous sedation and sedation combined with paralytics may present the greatest need for EEG-based sedation monitoring. These ICU sedation situations present challenges in accurate clinical assessment and monitoring, often resulting in over sedation and as well as under sedation. Over sedation in the ICU may result in decreases in quality of care; delays in treatment, longer stays, increased risk of ventilation and infection, and also impacts on neuromonitoring and triage of neurological complications, like stroke, seizure, and delirium. Under sedation, especially during the use of paralytics, may result in significant decreases in quality of care mainly from severe patient discomfort from poor pain management and psychological trauma. While there has been some use of EEG-based sedation monitoring outside in the ICU, its use has been limited due to difficulty of use, the lack of easy-to-understand readouts, and the resulting need for highly trained specialists to operate the EEG.

### Sedation and Delirium

The independent clinical assessment and treatment challenges for both sedation and delirium may be further complicated by the relationship that exists between patient sedation and delirium; over sedation and/or the use of particular sedatives may increase a patient's risk of delirium. Nonetheless, sedation may sometimes be a favored treatment method for certain presentations of delirium (i.e., hyperactive delirium) which may prolong the effects of other delirium sub-types. Furthermore, sedation, particularly over sedation, can mask the symptoms of delirium that are present at clinical recognition and limit the clinical feedback if a treatment is effective. As a result, there is a need to effectively monitor and detect both sedation and delirium levels of a patient.

### Detection and Monitoring System

### I. Signal Acquisition and Pre-Processing

### II. Signal Analysis

### III. Neurological Condition Detection and Output

### IV. Delirium Detection

### V. Post Neurological Condition Detection

### VI. Computer Systems

### I. Signal Acquisition and Pre-Processing

For ease of explanation, the figures and corresponding description below are described below with reference to analysis of signals representing brain activity (e.g., electroencephalography (EEG) signals) and/or heart activity (e.g., electrocardiography (ECG) signals) of a living subject. However, one of skill in the art will recognize that signals representing other bodily functions (e.g., an electromyography (EMG) signal, or an electronystagmography (ENG) signal, a pulse oximetry signal, a capnography signal, and/or a photoplethysmography signal) may be substituted, or used in addition to (e.g., in conjunction with), one or more signals representing brain activity and/or heart activity. In some variations, the signals are EEG signals analyzed to detect delirium in a patient.

A system for measuring bioelectrical signals may generally comprise one or more electrodes electrically coupled via corresponding conductive wires to a controller and/or output device. In other variations, the electrodes may be coupled to the controller and/or output device wirelessly. The electrodes may be contained within an electrode carrier system that is secured around the head of the patient. The electrode carrier system may be configured as a headband or incorporated into any number of other platforms or positioning mechanisms for maintaining the electrodes against the patient body. Individual electrode assemblies may be spaced apart from one another so that, when the headband is positioned upon the patient's head, the electrode assemblies may be aligned optimally for receiving EEG signals. In some variations, the electrode carrier system may be used to detect delirium in a patient.

In some variations, EEG signals from 10 electrodes may be combined. The locations of the electrodes may be, for example, Fp1, Fp2, F7, F8, T3, T4, T5, T6, O1, and O2. These electrodes may form 8 channels (Fp1-F7, F7-T3, T3-T5, T5-O1, Fp2-F8, F8-T4, T4-T6, and T6-O2, or any combination thereof). In other variations, EEG signals from 16 electrodes may be combined. The locations of the electrode may be, for example, Fp1, Fp2, F3, F4, F7, F8, C3, C4, P3, P4, T3, T4, T5, T6, O1, and O2. The use of 16 electrodes may be employed to generate long-field EEG channels useful to detect delirium. For example, EEG electrodes Fp1-F7, F7-T3, and T3-T5 may be used to generate a long-field channel Fp1-T5. This expanded EEG montage may be used for subsequent processing and prediction for delirium detection. Other long-field channels that may be generated include without limitation, Fp1-O1, Fp1-T5, F7-O1, Fp1-T3, F7-T5, T3-O1, Fp2-O2, Fp2-T6, F8-O2, Fp2-T4, F8-T6, T4-O2, Fp1-F8, Fp1-T4, Fp1-T6, Fp1-O2, F7-Fp2, F7-F8, F7-T4, F7-T6, F7-O2, T3-Fp2, T3-F8, T3-T4, T3-T6, T3-O2, T5-Fp2, T5-F8, T5-T4, T5-T6, T5-O2, O1-Fp2, O1-F8, O1-T4, O1-T6, Fp1-Fp2, and 01-O2.

The number of channels from which EEG signals are obtained and recorded may range from 1 to 45, including all values and sub-ranges therein. For example, the plurality of channels may include 1 channel, 2 channels, 3 channels, 4 channels, 5 channels, 6 channels, 7 channels, 8 channels, 9 channels, 10 channels, 11 channels, 12 channels, 13 channels, 14 channels, 15 channels, 16 channels, 17 channels, 18 channels, 19 channels, or 20 channels, 22 channels, 23 channels, 24 channels, 25 channels, 26 channels, 27 channels, 28 channels, 29 channels, 30 channels, 31 channels, 32 channels, 33 channels, 34 channels, 35 channels, 36 channels, 37 channels, 38 channels, 39 channels, 40 channels, 41 channels, 42 channels, 43 channels, 44 channels, or 45 channels. In one variation, an 8 channel EEG may be used to detect delirium. In another variation, a 16 channel EEG may be used to detect delirium.

The electrodes may be part of an electrode assembly and electrode carrier system, as mentioned above. The electrode carrier system may generally comprise an electrode body which is at least partially electrically conductive, one or more members (e.g., one or more tubular members) extending from the electrode body, each of the one or more members defining a lumen therethrough and a distal opening, a reservoir having a compressible structure and containing a conductive fluid or gel which is in fluid communication with the one or more members, and a backing supporting the electrode body and reservoir.

In some variations, the electrode carrier system may generally comprise an electrode body having one or more tubular members extending therefrom, each of the tubular members defining a lumen therethrough and a distal opening, a reservoir having a compressible structure which defines an internal volume and which is in fluid communication with the one or more tubular members, and a controller and/or output device which is in electrical communication with the electrode body, wherein the controller and/or output device is configured to receive electrical signals from the electrode assembly and record and/or output a corresponding response.

The electrode carrier system may generally comprise a backing secured around the head of a patient. The backing may be configured as a headband although the carrier system may be incorporated into any number of other platforms or positioning mechanisms for maintaining the electrodes against the patient body. The individual electrodes are spaced apart from one another so that when the headband is positioned upon the patient's head, the electrodes are aligned optimally upon the head for receiving EEG signals. The carrier system may have each of the electrodes electrically coupled via corresponding conductive wires extending from the backing and coupled, e.g., to a controller and/or output device. Although in other variations, the electrodes may be coupled to the controller and/or output device wirelessly.

The controller and/or output device may generally comprise any number of devices for receiving the electrical signals such as electrophysiological monitoring devices and may also be used in combination with any number of brain imaging devices, e.g., fMRI, PET, NIRS, etc. In one particular variation, the electrode embodiments described herein may be used in combination with devices such as those which are configured to receive electrical signals from the electrodes and process them.

In one variation, the electrode carrier system may comprise each of the electrodes enclosed within a reservoir which is pre-filled with a conductive gel or fluid. Each electrode may be configured into a flattened or atraumatic configuration which is contained within a respective reservoir and each reservoir may be formed of any number of flexible materials, e.g., silicone, polyurethane, rubber, etc., which can readily collapse. The electrodes may be coupled via conductive wires passing through a lumen defined through the backing separated from the electrodes by a substrate. Each reservoir may also respectively define one or more openings through which the conductive gel or fluid may be expelled.

Once the platform has been situated over the patients' head, the user may press upon each of the reservoirs such that the conductive fluid or gel flows through the openings and onto the skin of the patient. The conductive fluid or gel expelled through the openings may maintain fluid communication between the skin surface and the respective electrodes such that the detected electrical signals may be transmitted from the skin and to the electrodes. Moreover, because of the flexibility of the reservoirs, once the conductive fluid or gel has been expelled into contact with the skin surface, the backing may lie flat against the skin surface so that the patient may comfortably lay their head upon a surface while still maintaining electrical contact with the electrodes.

Another electrode variation may be comprised of one or more loops of conductive wire or ribbon which are able to readily bend or flex against the skin surface. The electrode carrier system may include a pressure release reservoir for containing the conductive fluid or gel, as described above, around each of the electrodes so that the conductive fluid or gel may be expelled around and within the one or more loops to ensure a conductive path.

In further variations of the electrode assembly, one or more tubular members may extend from the backing transversely. The tubular members may be each arranged in a circular pattern for each electrode and they may also define a lumen therethrough with an opening defined at each distal end. Each of the tubular members may be fabricated from a conductive metal which may retain its tubular shape when in use or which may be sufficiently thin and flexible to bend or yield when placed against the patient's skin surface. Alternatively, the tubular members may be fabricated from a flexible material which is coated or layered with a conductive material such that the members retain their flexibility. In either case, the conductive fluid or gel may be either contained within the tubular members or they may be retained within a pressure release reservoir, as described above, surrounding or in proximity to each electrode. Because of the tubular shape of the electrodes, they may readily pass through the patient's hair, if present, and into contact against the skin surface while maintaining electrical contact.

Yet another variation of an electrode embodiment may also utilize a pressure release reservoir filled with the conductive fluid or gel. The reservoir may be formed of a flexible material, e.g., silicone, polyurethane, rubber, etc., extending from the backing to form a curved or arcuate structure with one or more openings defined over the reservoir. These openings may remain in a closed state until a force is applied to the reservoir and/or backing which may urge the fluid or gel contained within to escape through the openings and into contact with the outer surface of the reservoir and underlying skin surface. The outer surface of the reservoir may have a layer of conductive material in electrical contact with the conductive wires so that once the fluid or gel has been expelled from within the reservoir and out onto the conductive material upon the reservoir outer surface and skin surface, electrical contact may be achieved.

The electrode carrier system in some instances may include an electrode body that may define one or more tubular members extending from the body such that the members project transversely away from the backing. The electrode body may be comprised of a conductive material such as a metal which may be rigid. However, in other variations, the body may be fabricated from a conductive material which is also flexible, e.g., conductive silicone, and/or from a flexible material, e.g., silicone, polyurethane, rubber, etc., which may be coated or layered with a conductive material such that the underlying tubular members retain their flexibility. In either case, the body may be secured to the backing such that the one or more openings are defined along the body and extending through the members are in fluid communication with a reservoir having a compressible housing. The reservoir may also be secured to the backing and contain a volume of conductive fluid or gel local to the electrode body.

The tubular members may be arranged in various patterns, e.g., a circular pattern, a uniform pattern, or in an arbitrary pattern. When the backing has been secured to the patient, the reservoir may be pressed or urged such that the fluid or gel contained within is expelled through each of the tubular members and into contact against the underlying skin surface through corresponding distal openings. The elongate nature of the members may enable them to pass readily through the patient's hair, if present, and into direct contact against the skin surface.

In another variation, an electrode carrier system having a tubular body may define one or more openings over its surface. The tubular body may have one or more tubular members which extend in a spiral or helical pattern away from the backing. The tubular members may define a lumen therethrough which extends from the tubular body and to a distal opening at its tip. The backing may further define a reservoir which contains a volume of conductive fluid or gel such that the body is in fluid communication with the reservoir. In some variations, the distal tips of the members may present a roughened surface for contacting the skin. The optionally roughened tips may be rotated or otherwise translated or moved across over the skin surface by the user to at least partially exfoliate the skin surface to facilitate electrical contact.

For example, a distal skin-contacting surface of the electrode assembly may be modified to prepare the skin surface to enhance electrical conductance (i.e., lower electrical resistance) between an electrically conductive portion of the electrode assembly and the skin when that electrically conductive portion is in physical contact with the skin. For example, the tissue-contacting surface(s) of the electrode assembly may be modified to have an abrasive surface, e.g., by coating with abrasive particulate; may be formed or molded to have protruding rigid features, e.g., bumps, ridges, or the like; and/or may be coated with a material that lowers the electrode connection impedance. Such sweeping and/or chemical coating of the tissue-contacting surface(s) of the electrode assembly over the target tissue location could scrub, dissolve and/or otherwise disrupt dead tissue and break-up scalp oil. In specific examples, at least a portions of a distal tissue-contacting surface of the electrode assembly, for example the distal surface(s) of at least some of the tubular members, comprise such surface features, surface coatings, surface treatments, or combination thereof to improve the quality of the electrode connection.

In another specific aspect of the present invention, an electrode assembly comprises an electrode body and one or more tubular members extending from the electrode body, typically from a bottom surface of the electrode body. Each tubular member has a distal tip, and at least some of the tubular members have a lumen with a distal opening in the distal tip. A reservoir containing a conductive fluid or gel is optionally disposed in the electrode body, and the electrode body is configured for dispensing the conductive fluid or gel from the reservoir through the lumen(s) and out of the distal opening(s) of the tubular member(s). Alternatively, in some embodiments, the conductive fluid or gel may be dispended onto or through the lumens of the tubular member using a syringe or other separate delivery device.

In specific embodiments, the electrode assembly will typically comprise at least two tubular members, and may comprise three tubular members, four tubular members, or even more. The tubular members will usually depend vertically downwardly from a bottom surface of the electrode body and will be specifically configured so that they may penetrate a patient's hair so that a distal tip of the tubular members will be able to engage and provide reliable electrical contact with a patient's scalp. The tissue engagement areas of the tubular members on bottom surface of the electrode body will usually be 50% or less of the area of the bottom surface, frequently being 30% or less of the area of the electrode body, and usually being at least 5% of the area of the bottom surface. Thus, the tissue engagement areas of the tubular members on bottom surface of the electrode body will usually be in a range from 5% to 50% of the area of the bottom surface, typically being in a range from 5% to 30% of the area of the bottom surface.

In most instances, the tubular members will extend from a generally planar bottom of the electrode body at a perpendicular angle. In other instances, however, the tubular members may extend at an angle anywhere in the range from 30° to 150° relative to the plane, typically being from 60° to 120° relative to the plane. In other instances, however, the tubular members may have other configurations, for example being configured in a helical shape so that they may penetrate hair to a patient's scalp by rotating the electrode assembly around a vertical axis.

In other specific embodiments of the present invention, the distal tips of at least some of the tubular members will have a skin preparation, e.g., tissue-roughening, surface. For example, the tissue-roughening surface may comprise an abrasive material, such as a grit or other abrasive particles, formed over at least a portion of the distal tip of the tubular member. In other instances, the surface-roughening may comprise surface features, such as ridges, bumps, grooves, and the like, formed over at least a portion of the distal tip which contacts the patient's skin.

The electrode body, and in particular the tubular members connected to the electrode body, may be formed at least partly from electrically conductive materials, such as metals, electrically conductive coatings, embedded wires, or electrically conductive polymers. In such instances, the electrode body and/or the tubular members will provide at least a portion of the electrical path needed to conduct biological currents from the tip of the tubular member(s) to an electrical terminal or other conductive connector on the electrode body as described below. In other instances, however, the electrode body and/or the tubular members may be formed primarily or even entirely from an electrically non-conductive material. In such instances, the electrically conductive fluid or gel will provide most or all of the electrically conductive path needed to deliver the biological current from the distal tip of the tubular member to the electrical terminal after such conductive fluid or gel has been distributed throughout the electrode body and tubular member.

The members may comprise a variety of geometries. In some instances, the tubular members may be generally cylindrical having a lumen extending therethrough. In other instances, however, the tubular members may be formed as “prongs” having a relatively broad tissue-contacting region along a curved “axis” at their distal tips. In many instances, the tissue-contacting regions of the prongs will be generally crescent-shaped so that they will follow a generally circular path as they are rotated against the patient's tissue.

The prongs and other members (e.g., tubular members) may have a port in their tissue-contacting surfaces for delivering the electrically conductive fluid or gel to the patient's skin. In some instances, ports may be formed in a generally flat bottom surface of the tubular members or prongs. In other instances, the ports may be connected to a channel or other distribution feature on the tissue-contacting surface of the prong or other tubular member. In still further specific embodiments, the ports for delivering the electrically conductive fluid or gel may be located in a recessed surface of the prong which may adjacent to a tissue-contacting lower surface of the prong or other tubular member.

While the electrode assemblies will usually comprise one or more members (e.g., prongs, tubular members) as just discussed, in some alternative embodiments, the electrode body may have a generally flat bottom free from tubular and other protruding members. The flat bottom may be configured to engage the skin and have openings to release a conductive fluid or gel in any of the ways described elsewhere herein for delivering the conductive fluid or gel. The tissue-contacting surface(s) of such flat bottoms may be modified in any of the ways discussed herein, e.g., roughened or textured, to have electrical conductivity with the target tissue surface(s).

In use, the plurality of electrodes may be placed on patient's scalp by placing a headband or other headgear around the patient's scalp. The headband carries a plurality of electrode assemblies, for example as described above, and distal tip(s) of one or more tubular members extending from at least some of the electrode assemblies may be engaged against scalp tissue. An electrically conductive fluid or gel may then be extruded from a reservoir disposed in at least some of the electrode assemblies so that the fluid or gel passes through the tubular members to form an electrically conductive path to the patient's scalp tissue. The plurality of electrode assemblies may then be connected to a controller and/or output device configured to receive low power biological current from the electrode assemblies. In some variations, at least some of the plurality of electrode assemblies have roughened surfaces that may be rotated in order to abrade scalp tissue adjacent the distal tip(s) of said one or more tubular members in order to lower contact resistance between the electrode assembly and the scalp tissue.

In some embodiments, signals corresponding to brain electrical activity are obtained from a human brain and correspond to electrical signals obtained from a single neuron or from a plurality of neurons. In some embodiments, sensors include one or more sensors affixed (e.g., taped, attached, glued) externally to a human scalp (e.g., extra-cranial sensor). For example, an extra-cranial sensor may include an electrode (e.g., electroencephalography (EEG) electrode) or a plurality of electrodes (e.g., electroencephalography (EEG) electrodes) affixed externally to the scalp (e.g., glued to the skin and using conductive gel to form electrical contact), or more generally positioned at respective positions external to the scalp Alternatively, dry electrodes can be used in some implementations (e.g., conductive sensors that are mechanically placed against a living subject's body rather than planted within the living subject's body or contacted through a conductive gel). An example of a dry-electrode is a headband with one or more metallic sensors (e.g., electrodes) that is worn by the living subject during use. The signals obtained from an extra-cranial sensor may sometimes be called EEG signals or time-domain EEG signals. In some cases, a sensor may be an accelerometer or an inertial measurement unit (IMU) that may measure the mechanical movement of the subject and/or the device (e.g., produce one or more electrical signals corresponding to mechanical movement of the subject and/or device). The system may be configured to utilize one or more sensors to aid in determining a potential neurological condition as described elsewhere herein.

### Neurological Condition Detection and Monitoring System—Data Module

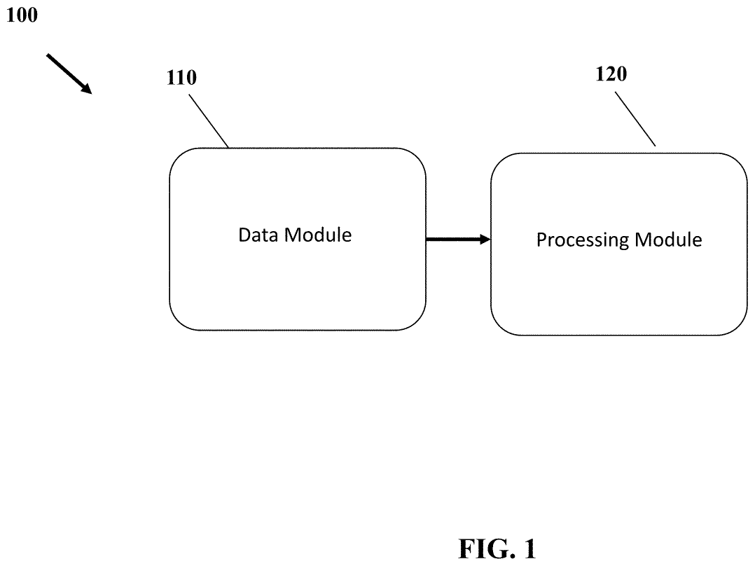

In an aspect, the present disclosure provides a neurological condition detection and monitoring system 100. As shown in FIG. 1, the system 100 may include a data module 110 configured to obtain data. The data obtained by the data module 110 may include a plurality of electroencephalography (EEG) signals collected from a subject. The data may also include non-EEG data. The non-EEG data may include blood pressure, heart rate, and/or motion data of the subject. The non-EEG data may be as described elsewhere herein.

In another aspect, the present disclosure provides a neurological condition detection and monitoring method. The neurological condition detection and monitoring method may include obtaining data. The data may include a plurality of electroencephalography (EEG) signals collected from a subject. The method may include processing the data to (1) detect and monitor one or more neurological conditions that the subject is experiencing or likely to experience, and (2) generate indications or assessments (i) for each neurological condition at an individual level and optionally (ii) between two or more related neurological conditions, wherein the one or more neurological conditions is selected from the group consisting of sedation, delirium, stroke and seizure.

The data module 110 may include a plurality of electrodes that are configured to be placed on different regions of the subject's head. The different regions may include frontal lobes, temporal lobes, and/or occipital lobes. The data module may also include a plurality of channels that multiplexes the EEG signals from the plurality of electrodes in each region and between the different regions. The electrodes may be used on the frontal lobes of the patient. The location of electrodes may be, for example, Fp1, Fp2, F7, F8, T3, T4, T5, T6, O1, O2 and channels (Fp1-F7, F7-T3, T3-T5, T5-O1, Fp2-F8, F8-T4, T4-T6, and T6-O2, or any combination thereof. In some cases, having electrodes at multiple locations may provide more coverage, more channels, more tolerance for noise or artifact from a specific channel, and capable of monitoring effects of different agents that affect different parts of the brain. In some cases, having electrodes at multiple locations may allow for more accurate or precise determination of neurological conditions. The number of channels provided by the electrodes may range, for example, between 1 and 45, as previously described herein. When delirium is to be detected, it may be beneficial to employ 8 channels or 16 channels. For example, when used for delirium detection, electrodes Fp1-F7, F7-T3, and T3-T5 may be used to generate a long-field channel Fp1-T5. In other variations for delirium detection, the long-field channels that may be generated include without limitation, Fp1-O1, Fp1-T5, F7-O1, Fp1-T3, F7-T5, T3-O1, Fp2-O2, Fp2-T6, F8-O2, Fp2-T4, F8-T6, T4-O2, Fp1-F8, Fp1-T4, Fp1-T6, Fp1-O2, F7-Fp2, F7-F8, F7-T4, F7-T6, F7-O2, T3-Fp2, T3-F8, T3-T4, T3-T6, T3-O2, T5-Fp2, T5-F8, T5-T4, T5-T6, T5-O2, O1-Fp2, O1-F8, O1-T4, O1-T6, Fp1-Fp2, O1-O2.

In some embodiments, the data module 110 may have one or more analog front ends configured to receive sensor EEG signals from sensors. The EEG signals may be preprocessed as described elsewhere herein. In some embodiments, a separate (e.g., independent) analog front end may be provided for interfacing with each of a set of sensors. In some embodiments, one or more analog front ends may be provided for interfacing with a set of EEG sensors.

### Neurological Condition Detection and Monitoring System—Processing Module

The system may include a processing module 120 in communication with the data module 110. The processing module 120 may be configured to process the data (e.g., EEG signals) to detect and monitor one or more neurological conditions that the subject is experiencing or likely to experience. The processing module 120 may generate indications or assessments (i) for each neurological condition at an individual level and optionally (ii) between two or more related neurological conditions. The indications or assessments may be presented to a user via a notification output module 290.

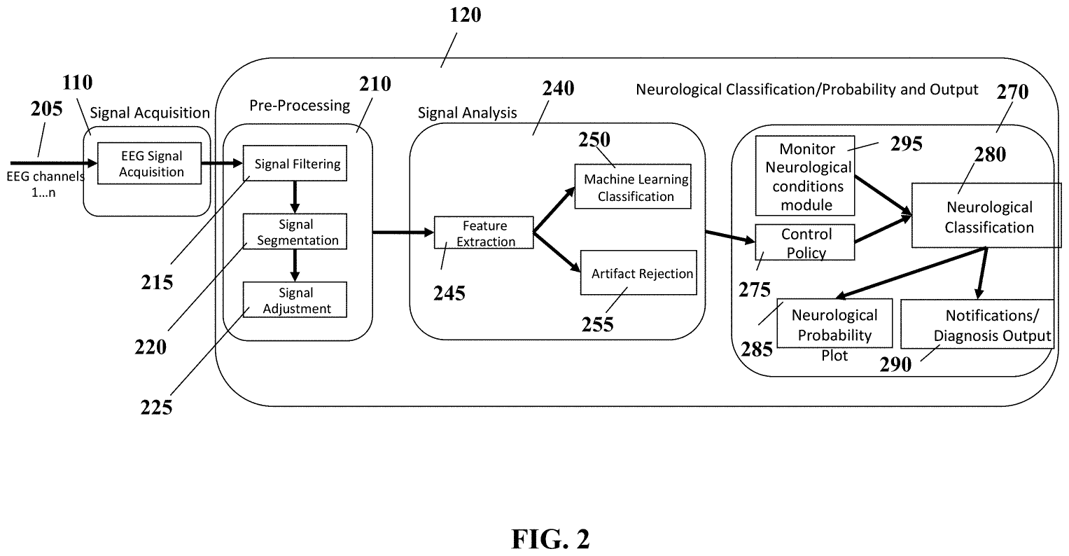

As shown in FIG. 2, the processing module 120 may be configured to process the data from the data module 110 to simultaneously detect and monitor the one or more neurological conditions in real-time. The one or more neurological conditions may relate to, for example, at least one of sedation, delirium, stroke and seizure. The two or more related neurological conditions may include sedation and/or delirium. The indications or assessments generated by the processing module may be indicative of a relationship or degree of correlation between sedation and delirium. The processing module may be configured to convert the data into one or more corollary assessment scores that are based at least on Riker Sedation-Agitation Scale (SAS), Richmond Agitation and Sedation Scale (RASS), Bispectral index monitor (BIS), and/or Confusion Assessment Method Intensive Care Unit (CAM-ICU), CAM-ICU-7, the Delirium Rating Scale Revised (DRS-R-98), the Intensive Care Delirium Screening Checklist (ICDSC), and/or one or more applicable scales for one or more indications. The processing module may be configured to convert the data into one or more corollary assessment scores that are based at least on the Ramsey Sedation Scale (RSS).

The processing module may be configured to convert the data into one or more corollary assessment scores that are based at least on Riker Sedation-Agitation Scale (SAS). The SAS scale may range from 1 to 7. One on the SAS scale may indicate unarousable wherein the subject has minimal or no response to noxious stimuli. The subject may not communicate or follow commands. Two on the SAS scale may indicate very sedated, wherein the subject may be aroused to physical stimuli but does not communicate or follow commands. The subject may move spontaneously. Three on the SAS scale may indicate sedated, wherein the subject is difficult to arouse but awakens to verbal stimuli or gentle shaking, follows simple commands but drifts off again. Four on the SAS scale may indicate calm and cooperative, wherein the subject is calm, easily arousable, and follows commands. Five on the SAS scale may indicate agitated, wherein the subject is anxious or physically agitated, calms to verbal instructions. Six on the SAS scale may indicate very agitated, wherein the subject requires restraint and frequent verbal reminding of limits, biting ETT. Seven on the SAS scale may indicate dangerous agitation, wherein the subject pulls at the ET tube, trying to remove catheters, climbs over bedrail, strikes at staff, thrashes side-to-side. In some cases, the subject is scored on the most severe degree of agitation displayed.

The processing module may be configured to convert the data into one or more corollary assessment scores that are based at least on Richmond Agitation and Sedation Scale (RASS). The RASS scale may range from −5 to +4. A negative five score on the RASS scale may indicate unarousable, wherein there is no response to voice or physical stimulation. A negative four score on the RASS scale may indicate deep sedation, wherein there is no response to voice, but movement or eye opening to physical stimulation from the subject. A negative three score on the RASS scale may indicate moderate sedation, wherein there is movement or eye opening to voice (but no eye contact) from the subject. A negative two score on the RASS scale may indicate light sedation, wherein the subject briefly awakens with eye contact to voice (<10 seconds). A negative one score on the RASS scale may indicate drowsy, wherein the subject is not fully alert, but has sustained awakening (eye-opening/eye contact) to voice (>10 seconds). A zero score on the RASS scale may indicate alert and calm. A positive one score on the RASS scale may indicate restless, wherein the subject is anxious, but movements are not aggressive or vigorous. A positive two score on the RASS scale may indicate agitated, wherein the subject has frequent non-purposeful movement, fights ventilator. A positive three score on the RASS scale may indicate very agitated, wherein the subject pulls or removes tube(s) or catheter(s); aggressive. A positive four score on the RASS scale may indicate combative, wherein the subject is overtly combative, violent, immediate danger to staff.

The processing module may be configured to convert the data into one or more corollary assessment scores that are based at least on bispectral index monitoring (BIS). The BIS sale may be from 0 to 100. A value of 0 may represent the absence of brain activity. A value of 100 may represent the awake state. BIS values between 40 to 60 may represent adequate general anesthesia for a surgery, values less than 40 may represent a deep hypnotic state.

The processing module may be configured to convert the data into one or more corollary assessment scores that are based at least on Ramsey Sedation Scale (RSS). The RSS scale may range from 1 to 6. A one on the RSS scale may indicate that the patient is anxious and agitated or restless, or both. A two on the RSS scale may indicate that the patient is co-operative, oriented, and tranquil. A three on the RSS scale may indicate that the patient responds to commands only. A four on the RSS scale may indicate that the patient exhibits brisk response to light glabellar tap or loud auditory stimulus. A five on the RSS scale may indicate that the patient exhibits a sluggish response to light glabellar tap or loud auditory stimulus. A six on the RSS scale may indicate that the patient exhibits no response.

The processing module may be configured to convert the data into one or more corollary assessment scores that are based at least on Confusion Assessment Method Intensive Care Unit (CAM-ICU) or CAM-ICU-7, the Delirium Rating Scale Revised (DRS-R-98), or the Intensive Care Delirium Screening Checklist (ICDSC). The CAM-ICU flowsheet may depend on the acute change or fluctuating course of mental status of the patient, inattention of the subject, altered level of consciousness of the subject, and disorganized thinking of the subject. CAM-ICU-7 provides a 0 to 7 scale for severity of delirium.

### Preprocessing of EEG Signals

In some embodiments, the method may include preprocessing the plurality of signals by segmenting the plurality of signals for each channel into a plurality of temporal data segments. As shown in FIG. 2, the processing module 120 may include a pre-processing module 210. In some embodiments, the method may include preprocessing the plurality of EEG signals by segmenting the plurality of EEG signals for each channel into a plurality of temporal data segments. FIG. 2 shows an illustration of the processing module 120. The processing module intakes EEG signals from a plurality of channels from the data module 110. The processing module may preprocess the EEG signals from a plurality of channels with a preprocessing module 210 configured to preprocess EEG signals. As shown in FIG. 2, the preprocessing module can include a signal filtering module 215, signal segmenting module 220, and signal adjustment module 225.

In some embodiments, the filtering module 215 may be configured to may filter EEG signals from the incoming set of channels from the EEG device module as described elsewhere herein. In some cases, preprocessing may be, for example, segmenting the EEG signals, filtering the EEG signals based on frequency, adjusting the EEG signals, or as described elsewhere herein, etc.

In FIG. 2, the signal segmentation module 220 can be configured to segment EEG signals. Each temporal data segment of the EEG signal (which may be referred to herein as a “temporal segment”) may be associated with a given time epoch. Each time epoch may be defined by a start time and a duration. Multiple EEG signals from different EEG electrodes may be segmented to have temporal segments that correspond the same time epoch, so that they may be, by way of example, analyzed subsequently to detect temporal correlations in their respective waveforms. For each corresponding time epoch, a cluster of neurological condition-positive classifications based on an analysis of features extracted from the corresponding temporal segments as described herein below may be indicative of one or more potential neurological conditions. The neurological conditions may be relative to delirium, seizure, sedation, stroke, and/or any combination thereof.

In some embodiments, the plurality of EEG signals may be segmented to between 1 to 100000 data segments. In some cases, the number of EEG data segments may depend on the duration of the EEG recordings. In some cases, the number of EEG data segments may be fixed regardless of the duration of the EEG recordings.

In some embodiments, each temporal data segment may have a duration of between about 1 second to 1 hour. In some cases, each temporal data segment may have a duration of between about 1 second to 30 seconds. In some cases, each temporal data segment may have a duration of between about 1 second to 10 seconds. In some cases, the duration of each temporal data segment may be fixed for the entire EEG recording. In some cases, the duration of each temporal data segment may be variable or adaptive during an EEG recording.

In some embodiments, the preprocessing of the plurality of EEG signals may comprise applying one or more filtering steps to the plurality of EEG signals over the plurality of channels. The preprocessing of the plurality of EEG signals may comprise using at least 1 filter, 2 filters, 3 filters, 4 filters, 5 filters, 6 filters, 7 filters, 8 filters, 9 filters, 10 filters, 15 filters or more. The preprocessing of the plurality of EEG signals may comprise using at most about 15 filters, 10 filters, 9 filters, 8 filters, 7 filters, 6 filters, 5 filters, 4 filters, 3 filters, 2 filters or less. The preprocessing of the plurality of EEG signals may comprise using anywhere between 1 to 15 filters, 1 to 10 filters, 1 to 5 filters, or 1 to 3 filters.

In some embodiments, the one or more filtering steps may be applied before, during, and/or after the segmentation of the plurality of EEG signals. One or more of the filtering steps may include, for example, a digital filter, an analogue filter, or a combination thereof. One or more of the filtering steps may include, for example, a bandpass filter, low-pass filter, a high-pass filter, a band-stop filter, an all-pass filter, a Kalman filter, an adaptive filter, or a notch filter, etc. In some cases, the low frequency cutoff of the filters may be between 0.1 Hz and 5 Hz. In some cases, the high frequency cutoff of the filters may be between 5 Hz and 200 Hz. In some cases, the notch filter frequency may match the local power line frequency. In some cases, the notch filter frequency may be 50 Hz or 60 Hz to match the local power line frequency.

For delirium detection, a 16-Channel EEG may be generated from raw waveforms recorded at a sampling rate of 250 Hz. The signals may be band-pass filtered between 0.5 and 40 Hz using a 5th order Butterworth filter. In some variations, subsequent high-pass filtering may be used for better removal of DC components for some of the feature calculations, as further described below. The filtered EEG data may then be segmented into window sizes (durations) ranging from about 1 second to about 10 minutes, including all values and sub-ranges therein. In some instances, the window size may be greater than 10 minutes. The window size may be about 1 second, about 2 seconds, about 3 seconds, about 4 seconds, about 5 seconds, about 10 seconds, about 15 seconds, about 20 seconds, about 25 seconds, about 30 seconds, about 35 seconds about 40 seconds, about 45 seconds, about 50 seconds, about 55 seconds, about 60 seconds, about 2 minutes, about 3 minutes, about 4 minutes, about 5 minutes, about 6 minutes, about 7 minutes, about 8 minutes, about 9 minutes, or about 10 minutes with an overlap of 0% to 95% (between consecutive windows). For example, in some variations, the filtered EEG data may be segmented into 15 second windows with 33% (5 second) overlap between consecutive windows. In other variations, the filtered EEG may be segmented into 10 second windows with no overlap, or 4 second windows with 75% (3 seconds) overlap, or 60 second windows with 50% (30 seconds) overlap.

### EEG Signal Adjustment

FIG. 2 shows a signal adjustment module 225 configured to adjust an EEG signal. In some embodiments, the method may adjust any EEG signal. Adjusting an EEG signal may include, for example, increasing and/or decreasing the amplitude of the EEG signal, adding or decreasing the noise level of the EEG signal, increasing and/or decreasing the time epoch the EEG signal, increasing and/or decreasing the intensity of the EEG signal, increasing and/or decreasing the signal frequency of the EEG signal, increasing and/or decreasing the voltage of the EEG signal, changing the morphology of the EEG signal (e.g. the shape of the EEG signal), increasing and/or decreasing the periodicity of the EEG signal, increasing or decreasing the synchrony of the EEG wave, spectral subtraction, standardizing etc.

In some cases, the EEG signal may be reduced. In some cases, the EEG signal may be down-sampled to a lower sampling frequency. For example, EEG data recorded at a sampling frequency of 500 Hz may be down sampled by a factor of 2 to 250 Hz.

In some cases, the EEG signal may be subjected to bit-width reduction. In some cases, the level of resolution at which the EEG signals are recorded may not be required by the method to achieve accurate neurological condition detections. In some cases, the bit-width reduction may reduce the EEG signal to a lower number of bits per sample through standard quantization of the EEG signal, for example, from 32 bits per sample to 12 bits per sample. In some cases, bit-width reduction may be advantageous if the method is to be implemented in a portable system, as it may be useful for reducing power consumption due to decreased processing load.

In some cases, spectral subtraction may be used to reduce the amount of additive noise in the EEG signal. In some cases, the noise may be caused by external surroundings. In some cases, the noise may be caused by the measurement equipment. In some cases, the noise may be caused by the user. In some cases, an average frequency spectrum of non-stroke EEG signal may be computed over a period of time to provide a base level estimate of the noise frequency spectrum. In some cases, as the EEG signals are recorded, the EEG signals may be converted to the frequency domain. In some cases, the average noise spectrum may then be subtracted from the EEG frequency spectrum. In some cases, the resulting spectrum and phase information from the original noisy signal may be combined. In some cases, the resulting spectrum may be transformed back into time domain to produce a de-noised signal.

In some embodiments, the EEG signal may be standardized by eliminating the effect of the montage that was used in gathering the EEG signals. In some cases, independent component analysis (ICA) or principal component analysis (PCA) methods may be used to provide the montage elimination. In some cases, the ICA or PCA method may separate the EEG signal into a set of sources independent of the montage used to record them. In some cases, using standardized EEG data may remove errors introduced by the varying practices of clinicians.

In some cases, a non-negative matrix factorization (NMF) method may be applied to each channel as a form of artifact removal. In some cases, the spectrum of the signal may be decomposed into the extracted bases to obtain weights. In some cases, the spectrum may be reconstructed using the bases of artifacts and the corresponding weights removed from the initial EEG signal. In some cases, independent component analysis (ICA) or principal component analysis (PCA) methods may be used for artifact reduction or removal.

### II. Signal Analysis

The processing module 120 may be configured to process the data to detect and analyze a plurality of features that are likely to be associated with the plurality of neurological conditions. The processing module may extract relevant new features from the data (e.g., EEG) that can be used to estimate, by way of example, Riker Sedation-Agitation Scale (SAS), Richmond Agitation and Sedation Scale (RASS), Bispectral index monitor (BIS), Confusion Assessment Method Intensive Care Unit (CAM-ICU), CAM-ICU-7, the Delirium Rating Scale Revised (DRS-R-98), the Intensive Care Delirium Screening Checklist (ICDSC), or other scales. The processing module 120 may be configured to use the plurality of features as inputs to train a machine learning algorithm for classifying different classes or severity relating to the plurality of neurological conditions.

### Feature Extraction

In FIG. 2, the processing module may comprise a signal analysis module 240. The signal analysis module 240 may comprise a feature extraction module 245 and a machine learning classification module 250. The feature extraction module 245 may be configured to take preprocessed measured data (e.g., EEG signals or temporal segments of the EEG signals of a given time epoch) from the preprocessing module 210 to build derived values (e.g., features).

The plurality of features extracted by feature extraction module 245 may include a plurality of time-domain features and frequency-domain features. The plurality of features may include a plurality of time-domain features and frequency-domain features of the data provided from the data module 110 and/or pre-processing module 210. The plurality of features may include brain asymmetry, amplitude variations, spatial and temporal correlations, coherences, or co-variations of two or more features. The plurality of features may be ranked and/or classified.

In some embodiments, feature extraction may start from an initial set of measured data (e.g., EEG signals or temporal segments of EEG signals of a given time epoch, etc.) and may build derived values (e.g., features) intended to be informative and non-redundant. In some cases, the feature extraction module may include extracting a plurality of features from each temporal data segment for each channel individually. In some cases, the feature extraction module may include extracting a plurality of features from each temporal data segment for all channels together. In some cases, the feature extraction module may include extracting a plurality of features from each temporal data segment of one or more groupings with each grouping consisting of one or more channels.

As shown in FIG. 2, the extracted features can be relayed to a machine learning classification module 250 that may be configured to analyze and classify the extracted features as described elsewhere herein. In some cases, feature extraction may facilitate the subsequent learning and generalization steps of a machine learning algorithm. In some cases, feature extraction may lead to better human interpretations. In some cases, feature extraction may be related to dimensionality reduction.

In some cases, when the input data (e.g., EEG signals) to the machine learning algorithm is too large to be processed and suspected to be redundant (e.g., the same measurement in both Hz and seconds, or the repetitiveness of a characteristic), the data can be transformed into a reduced set of features.

In some cases, determining a subset of the initial features may be called feature selection. In some cases, the selected features may be expected to contain the relevant information from the input data (e.g., EEG signals or temporal segments of the EEG signals). In some cases, the selected features may be expected to contain the relevant information from the input data so that the desired task can be performed by using this reduced representation instead of the complete initial data.

In some embodiments, feature extraction may involve reducing the number of resources required to describe a large set of data (e.g., EEG signals or temporal segments of the EEG signals). In some cases, analysis with a large number of variables may require a large amount of memory and computation power. In some cases, feature extraction may construct combinations of the variables to accurately describe the data with sufficient accuracy. In some cases, feature extraction may construct combinations of the variables to accurately describe the data with sufficient accuracy while preventing overfitting.

In some embodiments, results may be improved using constructed sets of application-dependent features. In some cases, the constructed sets may be built by an expert. In some cases, general dimensionality reduction techniques may be used. In some cases, general dimensionality reduction techniques may be, for example, independent component analysis, isomap, kernel PCA, latent semantic analysis, partial least squares, principal component analysis, multifactor dimensionality reduction, nonlinear dimensionality reduction, multilinear principal component analysis, multilinear subspace learning, semidefinite embedding, autoencoder, etc.

In some cases, a set of numeric features may be described by a feature vector. In some cases, a feature vector may be an n-dimensional vector of numerical features that represent some object, by way of example an EEG signal or a temporal segment of the EEG signal.

In some embodiments, data analysis software packages may provide for feature extraction. In some cases, data analysis software packages may provide for dimension reduction. In some cases, data analysis software packages may include programming environments such as MATLAB, SciLab, NumPy, or the R language, etc. In some cases, a programming language script may be used to extract features from EEG signals. In some cases, the programming language script may be, for example, MATLAB, Python, Java, JavaScript, Ruby, C, C++, or Perl, etc.

In some cases, the plurality of features may be intrinsic in the plurality of EEG signals or temporal segments thereof. Intrinsic may be a feature of an EEG signal that may be measured, for example, the amplitude of the EEG signal, the duration of the EEG signal, the variation of the EEG signal, the power of the EEG signal, the local maxima/minima of the EEG signal, the pattern of the EEG signal, the regularity of the EEG signal, the spectral power distribution of the EEG signal, or the frequency of the EEG signal, etc. In some cases, the plurality of features may be a measurement of the power of a signal within a particular frequency band. The frequency band may be, for example, from about 0 Hz to 100 Hz. In some cases, the power of a signal may be normalized to the total power. In some cases, the power of a signal may be a ratio of power between one or more frequency bands. In some cases, a feature may be a function performed on a signal to obtain a value. For example, a function may measure the root mean square (RMS) of a signal (e.g., EEG signal) to obtain the RMS value of the signal. In some cases, a feature may compare one signal (e.g., EEG signal) to one or more signals. In some cases, a feature may compare one or more signals (e.g., EEG signals) to one or more signals. In some cases, a feature may measure an attribute of a signal (e.g., EEG signal). In some cases, a feature may compare one or more attributes of a signal (e.g., EEG signal) with one or more attributes of a signal. An attribute may be, for example, an intrinsic property of the EEG signal. In some case, the feature of an EEG signal may be continuous and/or discrete in time.

In some cases, the plurality of features may include at least twenty different time and/or frequency domain features. In some cases, the plurality of features may include at most one thousand time and/or frequency features. In some cases, the plurality of features may include between about 10 features to 200 features. In some cases, the plurality of features may include between about 10 features to 100 features. In some cases, the plurality of features may include between about 10 features to 50 features.

In some cases, the plurality of features may include a plurality of discrete values associated with the time domain, frequency domain, time-frequency domain, information theory, and nonlinear-dynamics system theory features. In some cases, the plurality of features may include a plurality of discrete values associated with the time and/or frequency domain features. The plurality of features may include a plurality of continuous values associated with the time and/or frequency domain features or the morphology of the signal. In some cases, the plurality of features may also be brain asymmetry, amplitude variations, spatial and temporal correlations, coherences or co-variations of two or more features. In some cases, the plurality of features may also be frequency spectrum and characteristics of the EEG signal(s). In some cases, characteristics of the signal may include, jitter, skew, spread spectrum, time measurements, frequency measurements, etc. In some cases, analyzing the plurality of features may include ranking and/or classifying the plurality of features.

In some cases, the plurality of signals may be converted into a digital signal. In some cases, the plurality of signals may be converted into a digital signal and then an analog signal.

In some cases, the features may be sampled from a portion of the EEG signal. Features may be sampled from a portion of the EEG signal to reduce processing time and power required.

In some embodiments, a feature may be pertaining to a certain weight value. The weight value may give one feature a higher score for detecting a neurological condition or a particular neurological condition. The higher score may indicate that the feature may be more relevant in predicting one or more neurological conditions. The score may indicate that the feature may be more relevant in predicting a certain neurological condition. The score may indicate that the feature may be more relevant in predicting the severity of a particular neurological condition. The method may adjust the weight value of any feature at any given time. The method may adjust by increasing and/or decreasing the weight value of any feature at any given time.

In variations where delirium is to be detected, for each time window, e.g., each 4 second, each 10 second, each 15 second, each 30 second, or each 60 second window, features may be computed using time-frequency analysis of data recorded on individual channels to produce single-channel features. Additionally, a set of features may be computed to analyze signal interactions between pairs of channels, called multi-channel features. Exemplary single channel features may include power in different frequency bands (for example, Alpha, Beta, Delta, Theta, and Gamma), spectral properties, power ratios, amplitude characteristics and morphology features, entropy, variability and wavelet decomposition. Exemplary multi-channel features that may be computed to quantify inter-channel interactions may include correlation within and across hemispheres for different frequency bands (for example, Alpha, Beta, Delta, Theta, and Gamma), as well as spectral, amplitude and phase related correlations and synchrony measures.

An EEG window may be marked as an artifact if a predefined set of features cross certain threshold values. Further, if the most recently reported impedance on an electrode is higher than a preset threshold, the segment may also be marked as an artifact. If an EEG window is marked as an artifact, it is generally not used for subsequent analysis and predictions.

### Classification Using Machine Learning

In some embodiments, the method may include applying a machine learning algorithm to the plurality of features to perform a classification to one or more neurological conditions for each temporal data segment for each channel individually. In some cases, the machine learning classification module may include performing classification to one or more neurological conditions for each temporal data segment for all channels together. In some cases, the machine learning classification module may include performing classification to one or more neurological conditions for each temporal data segment of one or more groupings with each grouping consisting of one or more channels. FIG. 2 shows the machine learning classification module 250 that may take the features collected/extracted from the preprocessing step and classify the features. In some cases, the features may be extracted without a preprocessing step.

In some cases, machine learning algorithms may need to extract and draw relationships between features as conventional statistical techniques may not be sufficient. In some cases, machine learning algorithms may be used in conjunction with conventional statistical techniques. In some cases, conventional statistical techniques may provide the machine learning algorithm with preprocessed features.

In some embodiments, the plurality of features may be used by one or more machine learning algorithms to provide a classification for a temporal segment with respect to a neurological condition.

In some embodiments, a cluster of neurological indicative-positive classifications may comprise of between about 1 to 50 neurological indicative positive classifications. In some cases, a cluster of positive classifications may comprise of between 1 to 10 neurological indicative-positive calculations.

In some embodiments, for each corresponding time epoch, a cluster of delirium-positive classifications may be indicative of delirium and/or various levels of delirium. In some cases, a cluster of delirium indicative-positive classifications may comprise of between about 1 to 50 delirium indicative positive classifications. In some cases, a cluster of positive classifications may comprise of between 1 to 10 delirium indicative-positive calculations.

In some embodiments, for each corresponding time epoch, a cluster of sedation-positive classifications may be indicative of sedation and/or various levels of sedation. In some cases, a cluster of delirium indicative-positive classifications may comprise of between about 1 to 50 delirium indicative positive classifications. In some cases, a cluster of positive classifications may comprise of between 1 to 10 delirium indicative-positive calculations.

In some embodiments, for each corresponding time epoch, a cluster of seizure-positive classifications may be indicative of seizure and/or various levels of seizure. In some cases, a cluster of seizure indicative-positive classifications may comprise of between about 1 to 50 seizure indicative positive classifications. In some cases, a cluster of positive classifications may comprise of between 1 to 10 seizure indicative-positive calculations.

In some embodiments, for each corresponding time epoch, a cluster of stroke-positive classifications may be indicative of stroke and/or various levels of stroke. In some cases, a cluster of stroke indicative-positive classifications may comprise of between about 1 to 50 stroke indicative positive classifications. In some cases, a cluster of positive classifications may comprise of between 1 to 10 stroke indicative-positive calculations.

In some embodiments, the method may further comprise comparing the classifications sequentially across a plurality of time epochs on each channel. In some cases, before/after/during comparing the classifications sequentially across a plurality of time epochs on each channel, the classifications sequentially across a plurality of time epochs on each channel may be discarded. In some cases, a subset of the classifications may be discarded. In some cases, a subset of fewer than about 1 to 20 classifications may be discarded. In some cases, a subset of fewer than 3 classifications may be discarded. In some cases, a subset of fewer than 7 classifications may be discarded. In some cases, a subset of fewer than 10 classifications may be discarded. In some cases, a subset of fewer than 15 classifications may be discarded. In some cases, a subset of fewer than 20 classifications may be discarded.

In some embodiments, the subset of neurological indicative-positive classification may be discarded because, for example, they may be random readings, of low reliability, inaccurate classification, incorrect classification, calibration, system error, disconnected electrodes, artifactual signals, system interference, or other signals, etc.

In some embodiments, the subset of neurological indicative-positive classification may be discarded to, for example, conserve memory space, improve processing speed, reduce energy usage, reduce heat of the system, reduce calculation costs, save processing power, save processing time, increase reliability, or decrease random access memory usage, etc.

In some embodiments, the greater number of neurological indicative-positive classifications in a row may be indicative of high reliability. The greater the reliability of neurological indicative-positive classifications, the more accurate determination of detecting one or more neurological conditions in a patient. In some cases, the greater reliability of neurological indicative-positive classifications may be indicative of the machine learning algorithm accuracy, quality of data (EEG signals), or health status of the EEG detecting system, etc.

In some embodiments, a particular time epoch may be classified as associated with one or more neurological conditions if the temporal data segments for a subset of the plurality of channels are classified as neurological indicative-positive. In some cases, the subset may be at least 5%, 10%, 20%, 30%, 40%, 50% or more of the plurality of channels. In some cases, the subset may be at most about 50%, 40%, 30%, 20%, 10%, 5%, or less of the plurality of channels.

In some embodiments, a particular time epoch may be classified as associated sedation if the temporal data segments for a subset of the plurality of channels are classified as sedation indicative-positive. In some cases, the subset may be at least 5%, 10%, 20%, 30%, 40%, 50% or more of the plurality of channels. In some cases, the subset may be at most about 50%, 40%, 30%, 20%, 10%, 5%, or less of the plurality of channels.

In some embodiments, a particular time epoch may be classified as associated with delirium if the temporal data segments for a subset of the plurality of channels are classified as delirium indicative-positive. In some cases, the subset may be at least 5%, 10%, 20%, 30%, 40%, 50% or more of the plurality of channels. In some cases, the subset may be at most about 50%, 40%, 30%, 20%, 10%, 5%, or less of the plurality of channels.

In some embodiments, a particular time epoch may be classified as associated with stroke if the temporal data segments for a subset of the plurality of channels are classified as stroke indicative-positive. In some cases, the subset may be at least 5%, 10%, 20%, 30%, 40%, 50% or more of the plurality of channels. In some cases, the subset may be at most about 50%, 40%, 30%, 20%, 10%, 5%, or less of the plurality of channels.

In some embodiments, a particular time epoch may be classified as associated with seizure if the temporal data segments for a subset of the plurality of channels are classified as seizure indicative-positive. In some cases, the subset may be at least 5%, 10%, 20%, 30%, 40%, 50% or more of the plurality of channels. In some cases, the subset may be at most about 50%, 40%, 30%, 20%, 10%, 5%, or less of the plurality of channels.

In some embodiments, the classification may comprise assigning a probability value, by way of example between 0 and 1 or between 0 and 100, of a temporal segment being reflective of a subject having a neurological condition, for example, sedation, delirium, stroke, seizure, etc. In some embodiments, the classification may comprise assigning a severity value, by way of example between 0 and 7, of a temporal segment being reflective of the severity of the neurological condition, for example, sedation, delirium, stroke, seizure, etc. In some embodiments, the value assigned in the classification is a combined value reflecting both probability and severity.

In some embodiments, the classification may be a binary classification such as neurological condition-positive and neurological condition-negative. Alternatively, the score may be a classification of the temporal segment into one of three or more categories with respect to a neurological condition. A temporal segment may be classified as, by way of example, neurological condition-positive, neurological condition-negative, neurological condition-like, uncertain neurological condition activity, non-neurological condition activity, artifact, etc. The neurological condition classifications may relate to, for example, sedation, delirium, stroke, seizure, etc. A temporal segment may be classified as, for example, sedation-positive, sedation-negative, sedation-like, uncertain sedation activity, non-neurological condition activity, delirium-positive, delirium-negative, delirium-like, uncertain delirium activity, non-delirium activity, stroke-positive, stroke-negative, stroke-like, uncertain stroke activity, non-stroke activity, seizure-positive, seizure-negative, seizure-like, uncertain seizure activity, non-seizure activity, artifact, etc. A temporal segment may be classified as, for example, sedation-like and delirium-like. A temporal segment may be classified as, for example, sedation-positive, sedation-negative, sedation-like, uncertain sedation activity, delirium-positive, delirium-negative, delirium-like, uncertain delirium activity, and non-delirium activity.

The neurological condition classifications may pertain to one or more different neurological conditions. In some cases, the plurality of features may be classified into between 1 to 20 categories. In some cases, the plurality of features may be classified into between 1 to 10 categories. Individual categories may also be divided into sub-categories. For example, a neurological condition may be divided into one or more conditions that relate to, for example, sedation, delirium, stroke, seizure, etc. In some cases, a neurological condition may be divided into one or more subcategories that pertain to a particular corollary assessment score. For example, a sedation positive score may be classified as a certain value on the RASS scale, SAS scale, and/or RSS scale. For example, a delirium positive score may be classified as certain value on the CAM-ICU or CAM-ICU-7 scale. The values may be used to determine features that pertain to a particular score on the one or more corollary assessment scores. In another example, a temporal segment classified as stroke-positive may be further sub-divided into classifications based on the type of stroke. In some cases, stroke-positive classifications may be sub-divided by stroke type, stroke location or hemisphere, or stroke size or severity. Large vessel occlusion (LVO) strokes may be sub-divided into classifications based on the vessel where the occlusion is present.

In some embodiments, the method may include applying a machine learning algorithm to the plurality of features to perform a neurological multi-class classification (neurological condition-positive, neurological condition-negative, neurological condition-like, uncertain neurological condition activity, non-neurological condition activity, etc.) for each temporal segment for each channel.

In some embodiments, the method may include applying a machine learning algorithm to the plurality of features to perform a neurological multi-class classification (e.g., sedation vs delirium vs stroke vs seizure, sedation vs delirium vs stroke, delirium vs stroke vs seizure, sedation vs delirium, sedation vs stroke, sedation vs seizure, delirium vs stroke, delirium vs stroke, stroke vs seizure, stroke vs non-stroke, LVO vs non-LVO, etc.) for each temporal segment for each channel.

In some embodiments, the method may include applying a machine learning algorithm to the plurality of features to perform a neurological binary classification (e.g., neurological condition-positive vs neurological condition-negative) for each temporal data segment for each channel. In some embodiments, the one or more features collected may be discarded prior to or during machine learning classification or prior to categorizing.

In some embodiments, the method may include applying a machine learning algorithm to the plurality of features to perform a stroke multi-class classification (stroke-positive, stroke-negative, stroke-like, uncertain stroke activity, non-stroke activity, etc.) for each temporal segment for each channel. In some embodiments, the method may include applying a machine learning algorithm to the plurality of features to perform a stroke binary classification (e.g., stroke vs non-stroke, LVO vs non-LVO) for each temporal data segment for each channel. In some embodiments, the one or more features collected may be discarded prior to or during machine learning classification or prior to categorizing.

In some embodiments, the method may include applying a machine learning algorithm to the plurality of features to perform a delirium multi-class classification (e.g., delirium-positive, delirium-negative, delirium-like, uncertain delirium activity, etc.) for each temporal segment for each channel. In some embodiments, the method may include applying a machine learning algorithm to the plurality of features to perform a delirium binary classification (e.g., delirium vs. non-delirium) for each temporal data segment for each channel.

In some embodiments, the method may include applying a machine learning algorithm to the plurality of features to perform a sedation multi-class classification (sedation-positive, sedation-negative, sedation-like, uncertain sedation activity, etc.) for each temporal segment for each channel. In some embodiments, the method may include applying a machine learning algorithm to the plurality of features to perform a sedation binary classification (e.g., sedation vs non-sedation) for each temporal data segment for each channel.

In some embodiments, the method may include applying a machine learning algorithm to the plurality of features to perform a seizure multi-class classification (seizure-positive, seizure-negative, seizure-like, uncertain seizure activity, etc.) for each temporal segment for each channel. In some embodiments, the method may include applying a machine learning algorithm to the plurality of features to perform a sedation binary classification (e.g., seizure vs non-seizure) for each temporal data segment for each channel.

In some embodiments, a human may select, and discard features prior/during machine learning classification. In some cases, a computer may select and discard features. In some cases, the features may be discarded based on a threshold value. In some cases, the features may be discarded based on a one or more corollary assessment test and/or particular values within the corollary assessment test.

In some embodiments, any number of features may be classified by the machine learning algorithm. The machine learning algorithm may classify at least 10 features. In some cases, the plurality of features may include between about 10 features to 1000 features. In some cases, the plurality of features may include between about 10 features to 200 features. In some cases, the plurality of features may include between about 10 features to 100 features. In some cases, the plurality of features may include between about 10 features to 50 features. In some embodiments, the machine learning algorithm may be, for example, an unsupervised learning algorithm, supervised learning algorithm, or a combination thereof. The unsupervised learning algorithm may be, for example, clustering, hierarchical clustering, k-means, mixture models, DBSCAN, OPTICS algorithm, anomaly detection, local outlier factor, neural networks, autoencoders, deep belief nets, Hebbian learning, generative adversarial networks, self-organizing map, expectation—maximization algorithm (EM), method of moments, blind signal separation techniques, principal component analysis, independent component analysis, non-negative matrix factorization, singular value decomposition, or a combination thereof. The supervised learning algorithm may be, for example, support vector machines, linear regression, logistic regression, linear discriminant analysis, decision trees, k-nearest neighbor algorithm, neural networks, similarity learning, or a combination thereof. In some embodiments, the machine learning algorithm may comprise a deep neural network (DNN). The deep neural network may comprise a convolutional neural network (CNN). The CNN may be, for example, U-Net, ImageNet, LeNet-5, AlexNet, ZFNet, GoogleNet, VGGNet, ResNet18 or ResNet, etc. Other neural networks may be, for example, deep feed forward neural network, recurrent neural network, LSTM (Long Short Term Memory), GRU (Gated Recurrent Unit), Auto Encoder, variational autoencoder, adversarial autoencoder, denoising auto encoder, sparse auto encoder, Boltzmann machine, RBM (Restricted BM), deep belief network, generative adversarial network (GAN), deep residual network, capsule network, or attention/transformer networks, etc.

In some embodiments, the machine learning algorithm may be, for example, a Naïve Bayes classifier, linear regression, logistic regression, decision trees, random forests, rotation forests, K nearest neighbors (KNN), clustering, support vector machines (SVM), or neural networks. In some cases, the machine learning algorithm may include ensembling algorithms such as bagging, boosting, and stacking. The machine learning algorithm may be individually applied to the plurality of features extracted for each channel, such that each channel may have a separate iteration of the machine learning algorithm or applied to the plurality of features extracted from all channels or a subset of channels at once.

In some embodiments, the method may apply one or more machine learning algorithms. In some embodiments, the method may apply one or more one machine learning algorithms per channel.

In FIG. 2, the machine learning classification module 250 may comprise any number of machine learning algorithms. In some embodiments, the random forest machine learning algorithm may be an ensemble of bagged decision trees. In some cases, the ensemble of bagged decision trees may classify each temporal data segment for each channel as (1) neurological condition-positive or (2) neurological condition-negative. In some cases, the ensemble of bagged decision trees may classify each temporal data segment for each channel as (1) delirium-positive or (2) delirium-negative. In some cases, the ensemble of bagged decision trees may classify each temporal data segment for each channel as (1) sedation-positive or (2) sedation-negative. In some cases, the ensemble of bagged decision trees may classify each temporal data segment for each channel as (1) stroke-positive or (2) stroke-negative. In some cases, the ensemble of bagged decision trees may classify each temporal data segment for each channel as (1) seizure-positive or (2) seizure-negative.

The ensemble may be at least about 1, 2, 3, 4, 5, 10, 20, 30, 40, 50, 60, 70, 80, 90, 100, 120, 140, 160, 180, 200, 250, 500, 1000 or more bagged decision trees. The ensemble may be at most about 1000, 500, 250, 200, 180, 160, 140, 120, 100, 90, 80, 70, 60, 50, 40, 30, 20, 10, 5, 4, 3, 2 or less bagged decision trees. The ensemble may be from about 1 to 1000, 1 to 500, 1 to 200, 1 to 100, or 1 to 10 bagged decision trees.

In some embodiments, the method may include applying a machine learning classifier to any number of channels. The method may include applying a machine learning classifier to at least about 1, 2, 3, 4, 5, 6, 7, 8, 9, 10, 15, 20, 25, 50, 100, 500, 1000 or more channels. The method may include applying a machine learning classifier to at most about 1000, 500, 100, 50, 25, 20, 15, 10, 9, 8, 7, 6, 5, 4, 3, 2 or less channels. The method may include applying a machine learning classifier from about 1 to 1000, 1 to 100, 1 to 25, or 1 to 5 channels.

In some cases, the plurality of EEG signals may be collected over a plurality of channels. The machine learning algorithm may be individually applied to the plurality of features extracted for each channel, such that each channel has a separate iteration of the machine learning algorithm or applied to the plurality of features extracted from all channels or a subset of channels at once. Each channel may have at least about 1, 2, 5, 10, 25, 50, or more machine learning algorithms applied. Each channel may have at most about 50, 25, 10, 5, 2, or fewer machine learning algorithms applied.

In some embodiments, the method may include applying a machine learning classifier to a subset of channels. The subset of channels may be at least about 1%, 5%, 10%, 20%, 30%, 40%, 50% or more of the total set of channels. The subset of channels may be at most about 50%, 40%, 30%, 20%, 10%, 5%, 1% or less of the total set of channels. The subset of channels may be from about 1% to 50%, 1% to 40%, 1% to 30%, 1% to 20%, 1% to 10%, or 1% to 5% of the total set of channels.

In some embodiments, the machine learning algorithm may have a variety of parameters. The variety of parameters may be, for example, learning rate, minibatch size, number of epochs to train for, momentum, learning weight decay, or neural network layers etc.

In some embodiments, the learning rate may be between about 0.00001 to 0.1.

In some embodiments, the minibatch size may be at between about 16 to 128.

In some embodiments, the neural network may comprise neural network layers. The neural network may have at least about 2 to 1000 or more neural network layers.

In some embodiments, the number of epochs to train for may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 500, 1000, 10000, or more.

In some embodiments, the momentum may be at least about 0.1, 0.2, 0.3, 0.4, 0.5, 0.6, 0.7, 0.8, 0.9 or more. In some embodiments, the momentum may be at most about 0.9, 0.8, 0.7, 0.6, 0.5, 0.4, 0.3, 0.2, 0.1, or less.

In some embodiments, learning weight decay may be at least about 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or more. In some embodiments, the learning weight decay may be at most about 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.0001, 0.00001, or less.

In some embodiments, the machine learning algorithm may use a loss function. The loss function may be, for example, regression losses, mean absolute error, mean bias error, hinge loss, Adam optimizer and/or cross entropy.

In some embodiments, the parameters of the machine learning algorithm may be adjusted with the aid of a human and/or computer system.

In some embodiments, the machine learning algorithm may prioritize certain features. The machine learning algorithm may prioritize features that may be more relevant for detecting one or more neurological conditions, a particular neurological condition, a state of a subject associated with a neurological condition, delirium, seizure, sedation, stroke, and/or any combination thereof. The feature may be more relevant for detecting one or more neurological conditions if the feature is classified more often than another feature. The feature may be more relevant for detecting delirium, seizure, sedation, stroke, and/or any combination thereof if the feature is classified more often than another feature. In some cases, the features may be prioritized using a weighting system. In some cases, the features may be prioritized on probability statistics based on the frequency and/or quantity of occurrence of the feature. The machine learning algorithm may prioritize features with the aid of a human and/or computer system.

In some embodiments, one or more of the features may be used with machine learning or conventional statistical techniques to determine if a segment is likely to contain artifacts. FIG. 2 shows the artifact rejection module 255 which identifies segments containing artifacts. The identified artifacts may be a result of electrical interference, electrode instability or movement, subject movement, subject eye movement or blinking, subject chewing, subject muscle tensing, subject electrocardiographic artifact, etc. In some cases, movement sensors or other sensors may be used as an additional input to the artifact rejection module. In some cases, the identified artifacts can be rejected from being used in stroke classification. In some cases, the identified artifacts can be reduced, cancelled, or eliminated and the remaining signal may still be processed for stroke classification.

In some cases, the machine learning algorithm may prioritize certain features to reduce calculation costs, save processing power, save processing time, increase reliability, or decrease random access memory usage, etc.

### III. Neurological Condition Probability/Classification and Output

### Control Policy and Neurological Condition Probability/Classification

In some embodiments, the multi-neurological classification may include classifying each temporal data segment for each channel as (1) neurological condition-positive or (2) neurological condition-negative. In some embodiments, the multi-neurological classification may include classifying each temporal data segment for each channel as (1) sedation-positive or (2) sedation-negative. In some embodiments, the multi-neurological classification may include classifying each temporal data segment for each channel as (1) delirium-positive or (2) delirium-negative. In some embodiments, the multi-neurological classification may include classifying each temporal data segment for each channel as (1) seizure-positive or (2) seizure-negative. In some embodiments, the multi-neurological classification may include classifying each temporal data segment for each channel as (1) stroke-positive or (2) stroke-negative.

The multi-neurological classification may use machine learning algorithms as described elsewhere herein. The method may include aggregating the multi-neurological classifications for the plurality of temporal data segments for the plurality of channels over a moving time window. The method may include aggregating the sedation and delirium classifications for the plurality of temporal data segments for the plurality of channels over a moving time window. The classifications may be used to determine the level of sedation and/or delirium of a patient on a particular scale or assessment test.

The method may include aggregating the multi-delirium classifications and multi-sedation classifications for the plurality of temporal data segments for the plurality of channels over a moving time window. The method may include aggregating the multi-sedation classifications for the plurality of temporal data segments for the plurality of channels over a moving time window. The method may include aggregating the multi-delirium classifications for the plurality of temporal data segments for the plurality of channels over a moving time window. The method may include aggregating the multi-stroke classifications for the plurality of temporal data segments for the plurality of channels over a moving time window. The method may include aggregating the multi-seizure classifications for the plurality of temporal data segments for the plurality of channels over a moving time window. The method may include aggregating the multi-seizure classifications for the plurality of temporal data segments for the plurality of channels over a moving time window to aid in determining the level of sedation and/or delirium of a patient on a particular scale or assessment test.

The aggregated neurological classifications may be subjected to a control policy module 275 of the neurological condition probability/classification calculation and output module 270, as shown in FIG. 2. FIG. 2 shows the neurological condition probability/classification calculation and output module 270. As shown in FIG. 2, the neurological condition probability/classification calculation and output module 270 may comprise a control policy module 275, a neurological condition probability/classification calculation module 280, a neurological condition probability plot module 285, and a neurological condition notification module 290. The control policy module 275 may be configured to implement a control policy, the neurological condition probability/classification calculation module 280 may be configured to calculate a neurological condition probability, the neurological condition probability plot module 285 may be configured to plot neurological condition probability values, and the neurological condition notification module 290 may be configured to provide notifications and/or assessments as described elsewhere herein, respectively.

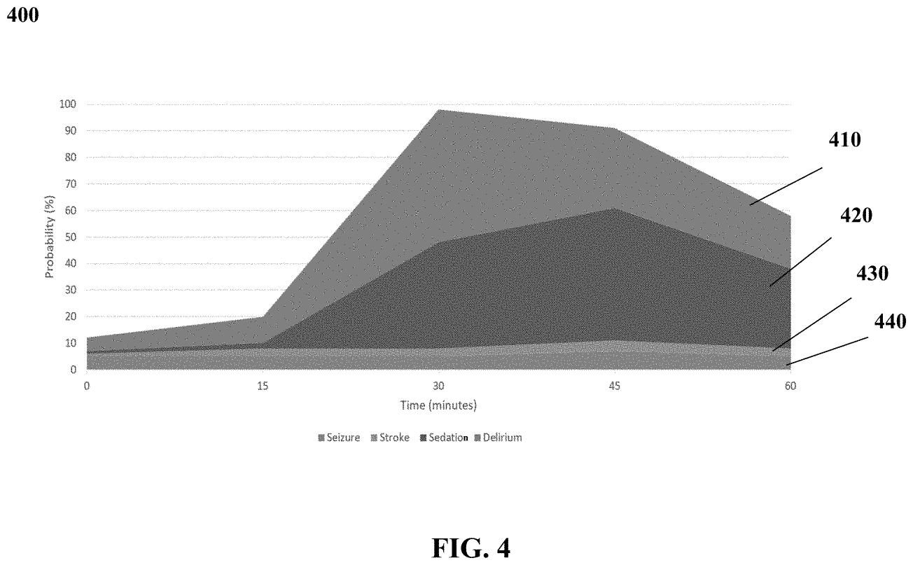

The processing module 120 may be further configured to generate a visual output. The visual output may include a graph that displays a probability/severity that the subject is experiencing one or more neurological conditions. The graphical representation may be a combination of a plurality of different temporal graphs corresponding to the plurality of neurological conditions. The graphical representation may include an overlay of the plurality of different temporal graphs. As shown in FIG. 4, the graphical representation 400 may include seizure 440, stroke 430, sedation 420, and delirium 410.

The processing module 120 may be configured to generate one or more corollary assessment scores that are indicative of the severity of one or more neurological conditions. The processing module 120 may be configured to generate a diagnostic output 290 based on the indications or assessments. The diagnostic output may include an aggregate wellness score or a graphical representation of the subject's brain state. The aggregate wellness score may be a combination of a plurality of discrete scores corresponding to the plurality of neurological conditions. The plurality of discrete scores may be combined based on different weights allocated to the plurality of neurological conditions. The processing module 120 may be configured to generate one or more corollary assessment scores that are indicative of one or more neurological conditions. The method may include generating one or more notifications when the patient has a wellness score below or above a particular wellness score.

In some cases, the moving window may have a period of time between 1 minute and 1 hour. In some cases, the period of time of the moving window may be dynamic or adjustable instead of fixed. In some cases, the period of time of the moving window may be dependent on the subject.

In some embodiments, a cluster of neurological condition-positive classifications on one or more channels may be subjected to a control policy module 275 to result in an overall determination of a neurological condition for the patient for a corresponding time epoch. In some embodiments, a cluster of sedation-positive and/or delirium-positive classifications on one or more channels may be subjected to a control policy module 275 to result in an overall determination of a sedation and/or delirium level for the patient for a corresponding time epoch.

The control policy may be a set of rules that result in an overall determination of neurological condition diagnosis or probability for the patient. The control policy may be a set of rules that result in an overall determination of delirium and/or sedation diagnosis or probability for the patient. The control policy may take a set of parameters as input and act on the set of parameters according to the set of rules to result in an overall determination of a neurological condition for the patient. The control policy may take a set of parameters as input and act on the set of parameters according to the set of rules to result in an overall determination of a sedation and/or delirium level for the patient. The set of rules may be as described elsewhere herein. The set of rules may be adjusted at any point of time to act on more parameters or to act on less parameters. The set of rules may be adjusted at any point of time to include more rules or to remove rules. The set of rules may be at least about 1, 2, 3, 4, 5, 6 7, 8, 9, 10, 15, 20, 25, 50, 100, 500, 1000, or more rules. The set of rules may be at most about 1000, 500, 100, 50, 25, 20, 15, 10, 9, 8, 7, 6, 5, 4, 3, 2, or less rules. The set of rules may be from about 1 to 1000, 1 to 500, 1 to 100, 1 to 25, 1 to 10, 1 to 5, or 1 to 3 rules.

In some embodiments, the input of parameters for the control policy may include, the quantity of classification of channels as neurological condition-positive, the quantity of classification of channels as neurological condition-negative, the classification of channels as neurological condition-positive, the classification of channels as neurological condition-negative, the classification of channels as neurological conditions, the corresponding time epoch, the quantity of channels, the machine learning algorithm used for classification, a moving window time length, the quality of the connection of each channel, information derived from EKG signals, information derived from EMG signals, information regarding the patient's demographics, information regarding the patient's current or previous condition, information regarding treatments or medications applied to the patient, information derived from movement sensors (e.g. an accelerometer or inertial measurement unit), etc.

In some embodiments, the control policy may have any number input of parameters. The control policy may have an input of at least about 1, 2, 3, 4, 5, 6, 7, 8, 9, 10, 11, 12, 13, 14, 15, 20, 25, 50, 100, 500, 1000, or more parameters. The control policy may have an input of at most about 1000, 500, 100, 50, 25, 20, 15, 14, 13, 12, 11, 10, 9, 8, 7, 6, 5, 4, 3, 2, or less parameters. The control policy may have an input from about 1 to 1000, 1 to 500, 1 to 100, 1 to 50, 1 to 25, 1 to 15, 1 to 10, or 1 to 5 parameters.

In some embodiments, the set of rules may dictate that the control policy discards the classification of a channel. For example, if the control policy receives an input of a single a neurological condition-positive classification for a corresponding time epoch, the set of rules may discard the neurological condition-positive classification for the corresponding time epoch. For example, if the control policy receives an input of a single a sedation-positive and/or delirium-positive classification for a corresponding time epoch, the set of rules may discard the delirium-positive and/or classification for the corresponding time epoch.

In some cases, the control policy may receive at least about 1, 2, 3, 4, 5, 6, 7, 8, 9, 10, 50, 100, 500, or more positive classifications and the set of rules may discard each positive classification for the corresponding time epoch. In some cases, the control policy may receive at most about 500, 100, 50, 10, 9, 8, 7, 6, 5, 4, 3, 2 or less positive classifications and the set of rules may discard each stroke-positive classification for the corresponding time epoch. In some cases, the control policy may receive from about 1 to 500, 1 to 100, 1 to 50, 1 to 10, or 1 to 5 positive classifications and the set of rules may discard each stroke-positive classification for the corresponding time epoch. The positive classification may pertain to neurological conditions. The positive classifications may pertain to sedation and/or delirium. The positive classifications may pertain to seizures. The positive classifications may pertain to strokes.

In some embodiments, the set of rules may dictate that the control policy output a neurological condition-positive classification for a set of channels corresponding to a time epoch. For example, if the control policy receives a set of four or more channels that each register a neurological condition-positive classification for the corresponding time epoch, the set of rules may output a neurological condition-positive classification for the corresponding time epoch. In some cases, the control policy may receive a set of neurological condition-positive classifications of at least about 1, 2, 3, 4, 5, 6, 7, 8, 9, 10, 15, 20, 25, 50, 100, 1000, or more channels, the set of rules may output a neurological condition-positive classification for the set of stroke-positive classifications for the corresponding time epoch. In some cases, the control policy may receive a set of neurological condition-positive classifications of at most about 1000, 100, 50, 25, 20, 15, 10, 9, 8, 7, 6, 5, 4, 3, 2, or less channels, the set of rules may output a neurological condition-positive classification for the set of neurological condition-positive classifications for the corresponding time epoch. In some cases, the control policy may receive a set of neurological condition-positive classifications from about 1 to 1000, 1 to 500, 1 to 100, 1 to 50, 1 to 25, 1 to 10, or 1 to 5, the set of rules may output a neurological condition-positive classification for the set of neurological condition-positive classifications for the corresponding time epoch.

In some embodiments, the set of rules may dictate that the control policy output a delirium-positive and/or sedation-positive classification for a set of channels corresponding to a time epoch. For example, if the control policy receives a set of four or more channels that each register a delirium-positive and/or sedation-positive classification for the corresponding time epoch, the set of rules may output a stroke-positive classification for the corresponding time epoch. In some cases, the control policy may receive a set of delirium-positive and/or sedation-positive classifications of at least about 1, 2, 3, 4, 5, 6, 7, 8, 9, 10, 15, 20, 25, 50, 100, 1000, or more channels, the set of rules may output a delirium-positive and/or sedation-positive classification for the set of delirium-positive and/or sedation-positive classifications for the corresponding time epoch. In some cases, the control policy may receive a set of delirium-positive and/or sedation-positive classifications of at most about 1000, 100, 50, 25, 20, 15, 10, 9, 8, 7, 6, 5, 4, 3, 2, or less channels, the set of rules may output a delirium-positive and/or sedation-positive classification for the set of delirium-positive and/or sedation-positive classifications for the corresponding time epoch. In some cases, the control policy may receive a set of delirium-positive and/or sedation-positive classifications from about 1 to 1000, 1 to 500, 1 to 100, 1 to 50, 1 to 25, 1 to 10, or 1 to 5, the set of rules may output a delirium-positive and/or sedation-positive classification for the set of delirium-positive and/or sedation-positive classifications for the corresponding time epoch.

In some embodiments, the method may include calculating the neurological condition probability/classification of the patient as the percentage of neurological condition-positive classifications within a specified period of time. In some embodiments, the method may include calculating the delirium and/or sedation probability/classification of the patient as the percentage of delirium-positive and/or delirium-positive classifications within a specified period of time.

As shown in FIG. 2, the neurological condition probability/classification calculation module 280 may be configured to calculate the neurological condition probability of the patient. The neurological condition probability/classification calculation module 280 may be configured to classify the neurological condition of the patient (e.g., delirium, sedation, seizure, stroke, etc.) In some cases, the period of time used for neurological condition probability/classification calculation may be between 1 minute and 1 hour. In some cases, the period of time used for neurological condition probability/classification calculation may be the entirety of the recording session. In some cases, the period of time used for neurological condition probability/classification calculation may be dynamic or adjustable instead of fixed. In some cases, the period of time used for sedation and/or delirium probability/classification calculation may be between 1 minute and 1 hour. In some cases, the period of time used for sedation and/or delirium probability/classification calculation may be the entirety of the recording session.

In some embodiments, the neurological condition probability may form a continuous output measured by calculating neurological condition probability for a moving window of time to result in a neurological condition probability/classification calculation for individual sequential periods of time. In some embodiments, the sedation and/or delirium probability may form a continuous output measured by calculating sedation and/or delirium probability for a moving window of time to result in a sedation and/or delirium probability/classification calculation for individual sequential periods of time. In some cases, the period of time of the moving window may be between 1 minute and 1 hour. In some cases, the period of time of the moving window may be dynamic or adjustable instead of fixed. In some cases, the sequential periods of time formed by the moving window may be overlapping. In some cases, sequential periods of time formed by the moving window may be non-overlapping. In some cases, the moving window may move in time increments between 1 second and 1 hour. In some cases, the moving window may pause or skip periods of time such that the resulting neurological condition probability/classification calculation values are not continuous or not sequential in time.

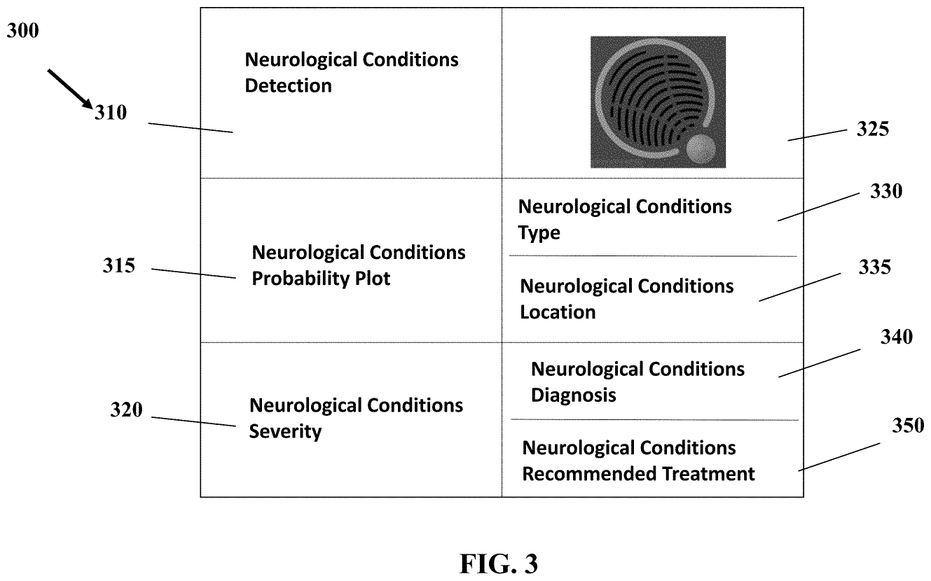

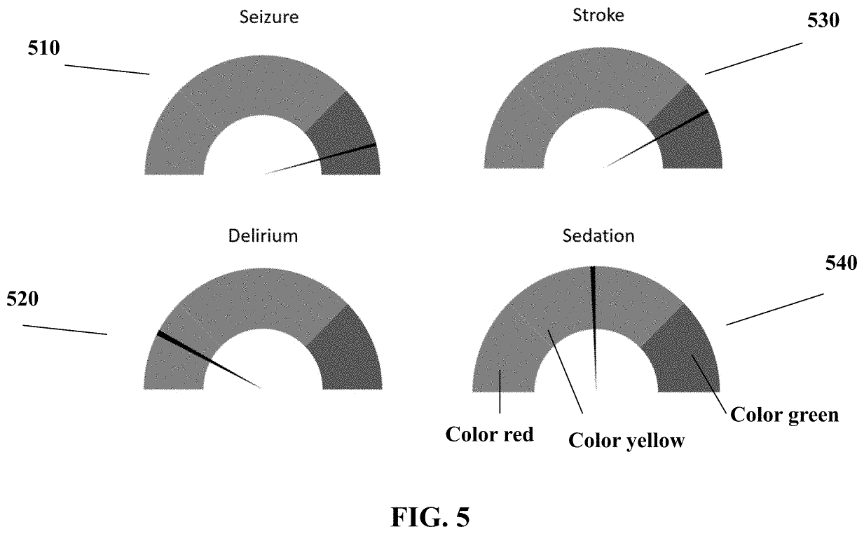

FIG. 2 shows a neurological condition probability plot module 285 configured to plot the neurological condition probability of a subject. As shown in FIG. 3, the neurological condition probability output 315 may be displayed to the user via an interface 300. The severity of the one or more neurological conditions 320 may also be displayed to the user. In some embodiments, the neurological condition probability output may display the one or more adjustable thresholds to the user on the time-series plot. In some embodiments, the neurological condition probability output may be displayed to the user as a time-series plot, bar graph, or chart etc. As shown in FIG. 4, the one or more neurological conditions may be illustrated as time vs probability 400. FIG. 4 shows the probability percentage of a patient for seizure 440, stroke 430, sedation 420, and delirium 410 over time in minutes. The time-series plot may plot the probability for seizure, stroke, sedation, delirium, or any combination thereof (e.g., seizure and delirium, sedation and delirium, stroke and sedation and delirium, etc.). As shown in FIG. 5, the one or more neurological conditions may be illustrated individually or with one another. A half circle meter plot may indicate the severity or probability of the neurological condition. The further right the indication line is, the greater the severity or probability of the neurological condition. The further left the indication line is, the less severe or lower probability of the neurological condition. As shown in FIG. 5, the system may include a half circle meter plot for seizure 510, stroke 530, delirium 520, and sedation 540.

In some embodiments, the time-series plot may be depicted in a certain color to note the threshold that has been passed or a particular severity has been reached. For example, if the probability of a delirium goes above a delirium threshold probability/severity value, a notification may be sent by the system. In some cases, the neurological condition probability plot module may display a wide variety of information, for example, the time period measured, the date, or the initial time acquisition, etc. In some cases, the neurological condition probability plot may be usable by a healthcare practitioner to assess the condition of the subject and determine a course of treatment. The neurological condition probability plot may also be usable by a healthcare practitioner to monitor the progression of the subject's condition over time or to monitor the effectiveness of courses of treatment.

FIG. 2 shows a notifications module 290 configured to generate notifications. In some embodiments, the method may include generating one or more notifications when neurological condition classifications have been made. In some embodiments, the method may include generating one or more notifications when sedation, delirium, seizure, and/or stroke classifications have been made. In some embodiments, the method may include generating one or more notifications when neurological condition-positive classifications have been made or when the neurological condition probability is equal to or exceeds one or more thresholds. In some cases, when the neurological condition probability/classification calculation value is equal to or exceeds an adjustable threshold value the system may display to a subject (e.g., patient) or user (e.g., healthcare practitioner, doctor, nurse, etc) a notification that the system has detected continuous neurological condition activity. The notification may also include any color. For example, the background of the screen displaying the notification may be red. The text of the notification may be any color, for example, white. The color of the background of the screen may correlate with the value of the neurological condition probability calculation. For example, if the neurological condition probability is equal to or above a certain threshold, the selected color for the background of the screen may indicate that the neurological condition probability is equal to or above a threshold. The color of the text of the notification may correlate with the value of the neurological condition probability calculation. For example, if the neurological condition probability calculation is equal to or above a certain threshold, the selected color for the text of the notification may indicate that the stroke probability is equal to or above a threshold.

In some embodiments, the method may include generating one or more notifications when sedation-, delirium-, seizure-, stroke-, or any combination thereof-positive classifications have been made or when the sedation-, delirium-, seizure-, stroke- or any combination thereof probability is equal to or exceeds one or more thresholds. In some cases, when the sedation-, delirium-, seizure-, stroke- or any combination thereof probability/classification calculation value is equal to or exceeds an adjustable threshold value the system may display to a subject (e.g. patient) or user (e.g. healthcare practitioner, doctor, nurse, etc.) a notification that the system has detected continuous sedation-, delirium-, seizure-, stroke- or any combination thereof activity.

The system may also display a wide variety of information to the subject or user in addition to the notification of detected continuous neurological condition activity. The system may display the neurological condition probability plot 315, severity of a neurological condition 320, location of the neurological condition within a subject 335, likelihood of a neurological condition to occur, classification of the neurological condition type 330, the time period for which the continuous neurological condition activity was detected (e.g., 7:40 pm to 7:50 pm), etc. The one or more notifications may be usable by a healthcare practitioner to assess the condition of the subject and determine a course of treatment. The notification may provide a diagnosis output to the healthcare practitioner. In some embodiments, the diagnosis output may include a neurological condition classification that is selected from a plurality of different neurological condition classes or neurological condition types. In some cases, the method may provide one or more neurological condition classifications described elsewhere herein. In some cases, the diagnosis output may provide symptoms pertaining to a neurological condition classification that correlate to a particular set of EEG signals. In some embodiments, the information in the diagnosis output may be useable to identify or detect one or more neurological condition pathologies.

In some embodiments, one or more notifications (e.g., diagnosis output) may be generated when the neurological condition probability value is equal to or exceeds one or more thresholds as described elsewhere herein. In some embodiments, one or more notifications (e.g., diagnosis output) may be generated when the neurological condition classification of the one or more features has been completed. In some cases, the one or more notifications may be generated in the form of visual, audio, and/or textual alerts. The device may include speakers 325 to provide audio notifications. In some cases, the one or more notifications may be delivered via networked communication technology such as the internet, telephone, facsimile, pager, short message service, etc. In some cases, the form, content, or delivery mechanism of the one or more notifications generated may depend on the neurological condition probability value. In some cases, the user may be able to select the form, content, or delivery mechanism of the one or more notifications generated.

In some embodiments, the neurological condition detection output may include an interface. The interface may provide indication of the EEG signal activity for the plurality of channels from the data module 110. The interface may display parameters that a user may adjust, for example, the time display, the scale, the high pass frequency, the low pass frequency, or the notch value, etc. The interface may also provide a neurological condition probability plot as described elsewhere herein. The interface may also provide neurological condition probability burden results over different time periods. The interface may also depict the stroke probability determination for each time segment. The interface may also provide a mechanism for the user to accept or reject the algorithm derived neurological condition probability determination or neurological condition classification. The interface may also provide a mechanism for the user to input their own determination of a neurological condition containing segments or neurological condition classification. In some cases, the neurological condition probability and/or neurological condition classification may be adjusted as a result of user entered information regarding neurological condition episodes. The displayed neurological condition probability/neurological condition classification and neurological condition notifications may be based solely on algorithm derived neurological condition determination, solely on user entered neurological condition determination, or on a combination of algorithm and user neurological condition determination.

In some embodiments, the neurological condition probability/classification calculation module may calculate neurological condition probability. The neurological condition notification module may output a notification if the neurological condition probability value crosses a threshold value. In some cases, notifications may be generated to a specific person that the method is programmed to notify. The threshold for notification may also be user adjustable.

In some embodiments, the neurological condition probability/classification calculation and output module may utilize criteria in addition to the neurological condition probability threshold to output a notification. In some cases, dynamic criteria may be applied with a combination of time based, neurological condition probability based, and other policies to determine if a notification is output.

In some embodiments, the neurological condition probability/classification calculation module may provide a probability value of a neurological condition to occur. The probability value may be provided as a percentage. The probability value may be provided as scaled-values (e.g., 1 to 10, 0 to 100, etc.). The probability values may be indicative of confidence of a neurological condition and/or the severity of the neurological condition. The probability value may be provided via a notification or a time-series plot as described elsewhere herein.

In some embodiments, the data may further include non-EEG data, wherein the non-EEG data comprises blood pressure, heart rate or motion data of the subject. In some cases, the EEG data and the non-EEG data are processed in a complementary or synergistic configuration to generate the diagnosis output or improve an accuracy of the diagnosis output. In some cases, the method is performed with the data collected from the subject in an external environment outside of a standard healthcare facility. In some cases, the external environment includes a pre-hospital location, a field environment or an ambulatory setting.

In some embodiments, the system may be coupled with other systems. In some cases, the systems may be eye trackers, movement sensors (e.g., an accelerometer or an inertial measurement unit), electromyography (EMG), electrocardiogram (ECG or EKG), etc. The collection of EEG data may be complemented with other inputs including, observed symptoms, other commonly collected biological inputs, such as heart signals (ECG or other heart rate monitors) and blood pressure, and passively collected movement measurements, such as from accelerometers and gyroscopes (for changes in movement, blood flow, and artifact detection), or questionnaire inputs collected from the user. Collection of the EEG and complementary input data may be rapidly used in an ambulance or other pre-hospital location, by practitioners in a hospital for quick triage in an emergency department (ED), for longer-term patient monitoring in an intensive care unit (ICU), and also perioperatively, before, during, and after surgical or other in-hospital procedures. For such procedures, separate or continuous recordings may be done in order to monitor for stroke and other changes, such as in sedation and patient health, brain degradation from any neurological or cardiovascular complications (edema, swelling, etc.), including stroke, and may also allow for more individualized, patient-based algorithm features, processing, tracking and baseline comparisons with baseline patient data (including but not limited to focal slowing, asymmetries, changes in delta and theta to delta ratios).

The complimentary non-EEG sensor data can be used for data selection or as independent features in the machine learning algorithm. The non-EEG data may be used to complement the EEG features to improve performance of the algorithm.

In some embodiments, the method may include transmitting the diagnostic output over one or more wired or wireless networks substantially in real-time to enable remote stroke management and care for the subject.

In some embodiments, the diagnostic output comprises a single diagnosis, a binary diagnosis, or a multi-tiered diagnosis associated with the neurological condition or the onset of the one or more neurological condition.

In some embodiments, the method may further be extendable and configured for diagnosis of acute traumatic brain injuries. In some cases, the EEG signals may include signal patterns that are associated with, or indicative of asymmetries in different areas and hemispheres of the subject's brain. In some embodiments, the EEG signals are passively collected using a set of electrodes worn on the subject's head. In some cases, the plurality of features includes at least fifty distinct features. In some cases, the plurality of features includes at least one hundred distinct features.

In some embodiments, the system may provide sedation monitoring across an array of drug agents, including, for example, Alfentanil, Desflurane, Fentanyl, Isoflurane, Nitrous Oxide, Propofol, Remifentanil, Sevoflurane, etc.

### IV. Delirium Detection

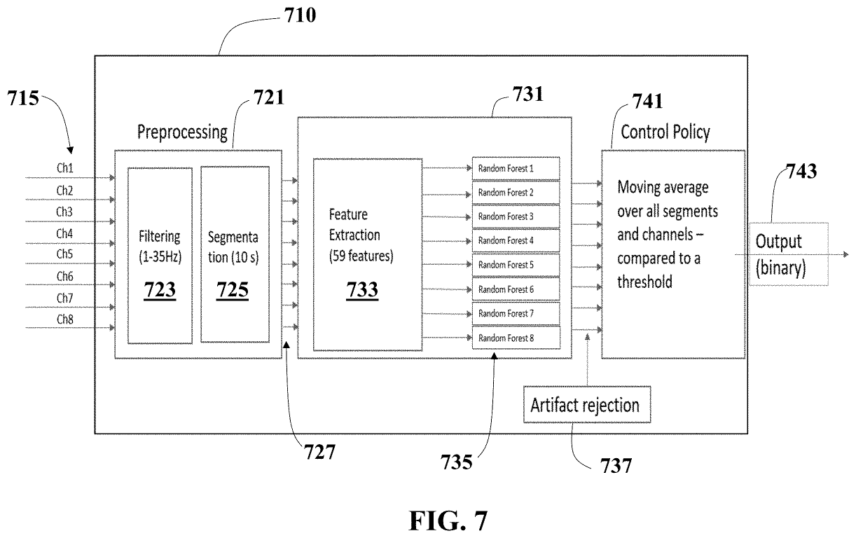

FIG. 7 schematically shows an exemplary delirium detection module 710 in accordance with certain embodiments of the disclosure that is intended to analyze previously acquired sections of adult (greater than or equal to 18 years) EEG recordings 715 in order to assist health service providers in the assessment of delirium. The EEG recordings comprise waveforms received from eight channels, each channel transmitting EEG recordings from one of eight EEG electrodes (not shown) placed on a subject's head. Preprocessing module 721 is configured to preprocess the incoming EEG recordings, and includes a filtering module 723 and a segmentation module 725 to process each of EEG recordings 715. The eight incoming EEG recordings are band-pass filtered between 1 and 35 Hz using a 10th order butterworth filter by filtering module 725, and divided into temporal segments, each segment having a 10-second duration.

The preprocessed temporal segments 727 of the EEG recordings are received to be further processed by machine learning module 731. The machine learning module comprises a feature extraction module 733 and a set of machine learning models 735. Feature extraction module 733 is configured to extract a predetermined set of features from each of the preprocessed temporal segments. Feature extraction module 733 may extract a plurality of different features, including one or more time-domain features, one or more frequency-domain features, and one or more correlation-based features, as provided above, e.g., in Tables 1 and 2, or other suitable features. The feature extraction module 733 as shown in FIG. 7 is configured to extract, by way of example, 59 features from each temporal segment. Examples of time-domain features extracted by feature extraction module 733 include but are not limited to: amplitude range, RMS of the amplitude, standard deviation of the amplitude, sharpness, area under the wave, number of local minima and/or maxima, peak amplitude, zero-crossings, RMS of the derivative of the signal, regularity. Examples of frequency-domain features extracted by feature extraction module 733 include but are not limited to: dominant frequency, dominant frequency power, leakage of signal outside of the dominant frequency, spectral entropy, power of signal in a given frequency band (e.g., alpha band, beta band, gamma band, delta band, or theta band). Examples of correlation-based features include a correlation of a channel with respect to the other channels, optionally on the same hemisphere of the brain.

Each one of machine learning models 735 is a random forest model that has been configured to classify a temporal segment as delirium-positive or delirium-negative based on the set of features extracted from the temporal segment. Each random forest model is an ensemble of 30 bagged decision trees, and is separately generated to classify temporal segments of EEG recording from one of the channels.

Each temporal segment may also be evaluated by artifact rejection module 737. A combination of some of the features extracted by feature extraction module 733 for each of the temporal segment is used by artifact rejection module 737 to determine if a given temporal segment comprises an artifactual signal or an excess of artifacts in the signal and should be excluded from further analysis to contribute to the final delirium assessment. A given EEG electrode typically operates at a characteristic impedance or within an impedance range when the electrode is in working order, and an impedance measurement that deviates from the impedance or impedance range indicates a defective or damaged electrode. Artifact rejection module 737 may also use an impedance measurement of each electrode to determine whether to exclude the temporal segment from further analysis.

The respective outputs of each of the random forest models 735 within a time window, which may encompass one or multiple successive time epochs, are combined and processed against a pre-defined set of rules, schematically shown as control policy 741, to determine an overall delirium score of a subject. The overall delirium score may be transmitted to other modules, devices, or components of a system as output 743 of the control policy. By way of example, the overall delirium score may be a value that is based on a percentage of temporal segments within the time-window that are delirium positive, such that a higher percentage of the temporal segments being classified as delirium-positive results in a higher delirium burden. The time-window may be a moving time-window, such that output 743 of overall delirium score is a trace or plot of the overall delirium score over time, and/or is dynamically updated. Alternatively or in addition, output 743 may comprise an alert that is generated when the overall delirium score exceeds a predetermined threshold.

When module 710 was used in a pilot study, the algorithm employed for delirium detection achieved clinical success, resulting in a sensitivity of about 95% and specificity of about 92% in the detection of delirium, as further described in Example 1. Additionally, continuous monitoring for delirium was achieved as soon as the electrodes began to record EEG waveforms. By providing continuous monitoring for delirium, module 710 may optimize delirium treatment and in turn, reduce the duration of delirium.

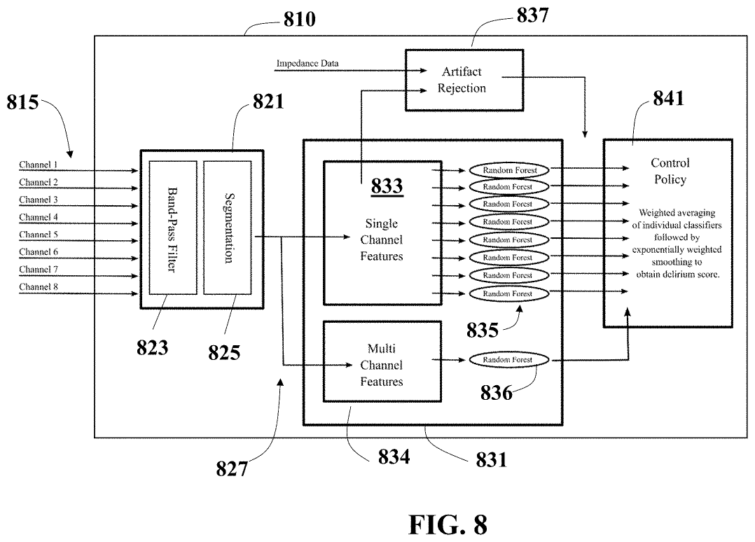

FIG. 8 schematically shows an alternative exemplary delirium detection module 810 in accordance with certain embodiments of the disclosure that is intended to analyze previously acquired sections of adult (greater than or equal to 18 years) EEG recordings 815 in order to assist health service providers in the assessment of delirium. The EEG recordings comprise waveforms received from eight channels, each channel transmitting EEG recordings from one of eight EEG electrodes (not shown) placed on a subject's head. Preprocessing module 821 is configured to preprocess the incoming EEG recordings, and includes a filtering module 823 and a segmentation module 825 to process each of EEG recordings 815. The eight incoming EEG recordings are band-pass filtered between 0.5 Hz and 40 Hz using a 5th order Butterworth filter by filtering module 823, then divided into temporal segments by segmentation module 825. Each temporal segment has a duration of 60 seconds. In addition, the first half (30 seconds) of the temporal segment overlaps with the previous temporal segment, and the second half of the temporal segment overlaps with the subsequent temporal segment.

The preprocessed temporal segments 827 of the EEG recordings are received to be further processed by machine learning module 831. The machine learning module comprises a single-channel feature extraction module 833 and a multi-channel feature extraction module 834. Single-channel feature extraction module 833 may be configured to extract a predetermined set of features from each of the preprocessed temporal segments 827. Single-channel feature extraction module 833 may extract a plurality of different features, including one or more time-domain features and one or more frequency-domain features. Examples of time-domain features extracted by feature extraction module 833 include but are not limited to: amplitude range, RMS of the amplitude, standard deviation of the amplitude, sharpness, area under the wave, number of local minima and/or maxima, peak amplitude, zero-crossings, RMS of the derivative of the signal, regularity. Examples of frequency-domain features extracted by feature extraction module 833 include but are not limited to: dominant frequency, dominant frequency power, leakage of signal outside of the dominant frequency, spectral entropy, power of signal in a given frequency band (e.g., alpha band, beta band, gamma band, delta band, or theta band).

Multichannel feature extraction module 834 is configured to extract a predetermined set of multi-channel features that quantify a degree of correlation between pairs of temporal segments from different EEG signals corresponding to a given time epoch. Unlike single channel features that characterize a given temporal segment from an EEG signal received from one channel, multichannel features characterize inter-channel interactions within a given time epoch. Examples of multichannel features includes but are not limited to: an average correlation coefficient for the EEG signal waveform received from pairs of electrodes within and/or across hemispheres (the waveforms may be filtered to isolate signals within alpha, beta, gamma, delta, or theta frequency bands), an average peak correlation (measured over different lags) within and/or across hemispheres, an average lag at which peak correlation is observed within and/or across hemispheres, and an average correlation coefficient of a power spectrum within and/or across hemispheres.

Machine learning module 831 further comprises a set of single-channel machine learning models 835 configured to receive and process single-channel features to provide a delirium probability for each temporal segment, and a multichannel machine learning model 836 configured to process multichannel features to provide a delirium probability for each time epoch. Each of the single-channel machine learning models 835 is a random forest model that is an ensemble of 30 binary decision trees (classifying an EEG window to be delirium positive or negative), and is separately generated for one of the channels to evaluate temporal segments of EEG recordings. Each model is trained using random under-sampling on the individual channels to predict a delirium probability for a given temporal segment.

Multichannel machine learning model 836 is a boosted random forest model that is trained on the multi-channel features for predicting delirium probabilities for a given time epoch. Each boosted random forest model comprises an ensemble of 50 binary decision trees (classifying an EEG window to be delirium positive or negative).

Each temporal segment may also be evaluated by artifact rejection module 837. A combination of some of the features extracted by single-channel feature extraction module 833 for each of the temporal segments is used by artifact rejection module 837 to determine if a given temporal segment comprises an artifactual signal or an excess of artifacts in the signal and should be excluded from further analysis to contribute to the final delirium assessment. Artifact rejection module 837 may also use an impedance measurement of each electrode to determine whether to exclude the temporal segment from further analysis.

The respective outputs of each of the single-channel machine learning models 835 and multichannel machine learning model 836 within a time window are combined and processed against a pre-defined set of rules, schematically shown as control policy 841, to determine an overall delirium score of a subject. The time window may encompass one or multiple successive time epochs.

The overall delirium score determined by control policy 841 may be transmitted to other modules, devices, or components of a system. By way of example, the overall delirium score may be a value that is a weighted combination of the delirium probabilities determined by single-channel machine learning models 835 and multichannel machine learning model 836.

The time-window may be a moving time-window, such that the overall delirium score may be presented as a trace or plot of the overall delirium score over time, and/or dynamically updated. Alternatively or in addition, control policy 841 may be configured to generate an alert when the overall delirium score exceeds a predetermined threshold.

In certain embodiments, delirium detection module 710 or delirium detection module 810 may be comprised in processing module 120 as shown in FIG. 2 and described herein above. In certain embodiments, delirium detection module 710 or delirium detection module 810 may operate within processing module 120 along with other detection modules the are configured to assess one or more other neurological conditions, such as but not limited to sedation, stroke, or seizure.

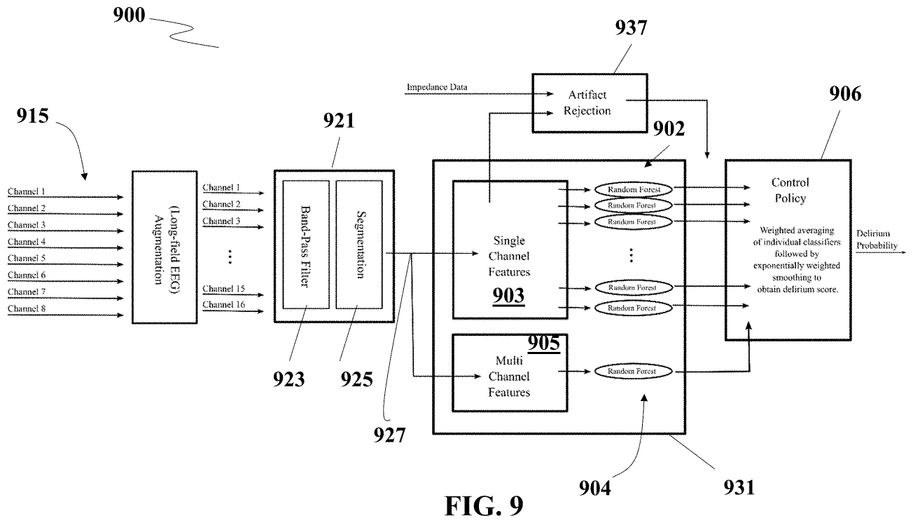

FIG. 9 schematically shows another exemplary delirium detection module 900 for analyzing previously acquired sections of EEG recordings 915 in order to assist health service providers in the assessment of delirium using machine learning model 931. The EEG recordings may be obtained from a plurality of electrodes that may be coupled to or incorporated into a headband, headgear, or other apparatus configured to place the electrodes on or around the head of the patient. In some variations, the EEG recordings are obtained from 10 electrodes coupled to or incorporated into a headband. In other variations, the EEG recordings are obtained from 16 electrodes coupled to or incorporated into a headband. Similar to the module 810 in FIG. 8, the delirium detection module 900 may include a preprocessing module 921 configured to preprocess the incoming EEG recordings. The preprocessing module 921 may include a filtering module 923 and a segmentation module 925 to process each of EEG recordings 915. The incoming EEG recordings may be band-pass filtered between 0.5 Hz and 40 Hz using a 5th order Butterworth filter by filtering module 923, then divided into temporal segments by segmentation module 925 to generate preprocessed temporal segments 927. Each temporal segment may have a duration of 60 seconds.

The preprocessed temporal segments 927 of the EEG recordings are received to be further processed by machine learning module 931. The machine learning module 931 may comprise a single-channel feature extraction module 903 and a multi-channel feature extraction module 905. Single-channel feature extraction module 903 may be configured to extract a predetermined set of features from each of the preprocessed temporal segments 927. Single-channel feature extraction module 903 may extract a plurality of different features, including one or more time-domain features and one or more frequency-domain features. Examples of time-domain features extracted by feature extraction module 903 include but are not limited to: amplitude range, RMS of the amplitude, standard deviation of the amplitude, sharpness, area under the wave, number of local minima and/or maxima, peak amplitude, zero-crossings, RMS of the derivative of the signal, and regularity. Examples of frequency-domain features extracted by feature extraction module 903 include but are not limited to: dominant frequency, dominant frequency power, leakage of signal outside of the dominant frequency, spectral entropy, power of signal in a given frequency band (e.g., alpha band, beta band, gamma band, delta band, or theta band). The plurality of different features that are extracted may also include power in different frequency bands (for example, alpha, beta, delta, theta, and gamma), spectral properties, power ratios, amplitude characteristics and morphology features, entropy, variability and wavelet decomposition.

Multichannel feature extraction module 905 may be configured to extract a predetermined set of multi-channel features that quantify a degree of correlation between pairs of temporal segments from different EEG signals corresponding to a given time epoch. Unlike single channel features that characterize a given temporal segment from an EEG signal received from one channel, multichannel features characterize inter-channel interactions within a given time epoch. Examples of multichannel features include but are not limited to: an average correlation coefficient for the EEG signal waveform received from pairs of electrodes within and/or across hemispheres (the waveforms may be filtered to isolate signals within alpha, beta, gamma, delta, or theta frequency bands), an average peak correlation (measured over different lags) within and/or across hemispheres, an average lag at which peak correlation is observed within and/or across hemispheres, and an average correlation coefficient of a power spectrum within and/or across hemispheres. In some variations, the multi-channel features that may be computed to quantify inter-channel interactions may include correlation within and across hemispheres for different frequency bands (for example, alpha, beta, delta, theta, and gamma), as well as spectral, amplitude and phase related correlations and synchrony measures. Multichannel machine learning model 905 may be a boosted random forest model that is trained on the multi-channel features for predicting delirium probabilities for a given time epoch. Each boosted random forest model may comprise an ensemble of 50 binary decision trees (classifying an EEG window to be delirium positive or negative).

In the model 931 of FIG. 9, 16 (same as the number of EEG channels) boosted random forests 902 may be trained using random under-sampling on the individual channel features to predict the delirium probability for each 60 second EEG window. Simultaneously, a boosted random forest 904 may be trained on the multi-channel features for predicting delirium probabilities. Each boosted random forest may consist of an ensemble of 50 binary decision trees (classifying an EEG window to be delirium positive or negative). A weighted combination of the probability measures obtained from the random forest models may be non-linearly combined to obtain a delirium score between 0 (indicating a low delirium probability) and 1 (indicating a high delirium probability). An exponentially weighted smoothing filter may be applied to the delirium probability score to produce a single value (also between 0 and 1) which can be used as a diagnostic tool for delirium.

Boosting may increase the emphasis when the model has difficult learning, enabling overall better prediction capability for the models. At the same time, random under-sampling of the data (e.g., 80% of the minority class available for training) may allow selection of a subset of the data such that the number of delirium positive and negatives visible to the model for training may be equalized.

Each temporal segment may also be evaluated by artifact rejection module 937. A combination of some of the features extracted by single-channel feature extraction module 903 for each of the temporal segments may be used by artifact rejection module 937 to determine if a given temporal segment comprises an artifactual signal or an excess of artifacts in the signal and should be excluded from further analysis to contribute to the final delirium assessment. Artifact rejection module 937 may also use an impedance measurement of each electrode to determine whether to exclude the temporal segment from further analysis.

The respective outputs of the single-channel machine learning model 903 and multichannel machine learning model 905 within a time window may be combined and processed against a pre-defined set of rules, schematically shown as control policy 906, to determine an overall delirium score of a subject. The time window may encompass one or multiple successive time epochs.

The overall delirium score determined by control policy 906 may be transmitted to other modules, devices, or components of a system. By way of example, the overall delirium score may be a value that is a weighted combination of the delirium probabilities determined by single-channel machine learning model 903 and multichannel machine learning model 905.

### V. Post Neurological Condition Detection

In some embodiments, the method may include generating one or more notifications as described elsewhere herein.

In some embodiments, the method may provide a user a response to minimize or prevent the detected neurological condition. The method may provide a response to minimize or reduce the risk of the onset of the neurological condition. In some cases, a therapeutic may be delivered to the subject to prevent and/or mitigate the predicted neurological condition. In some cases, the method may adjust the quantity of therapeutic delivered to the subject.

### VI. Computer Systems

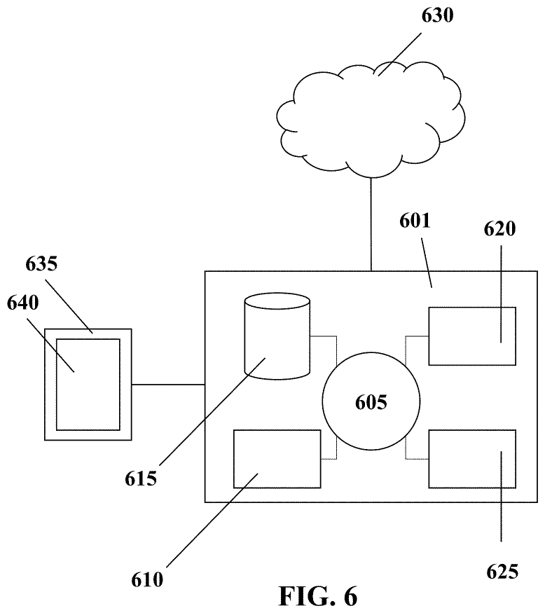

The present disclosure provides computer systems that are programmed to implement methods of the disclosure, including the control of the multi-detection system, control hardware components, receive and process data, interface with a user, etc. FIG. 6 shows a computer system 601 that is programmed or otherwise configured to operate and/or control the data module and the processing module. The computer system 601 can regulate various aspects of the present disclosure, such as, for example, determining neurological conditions, classification of neurological conditions, classification of EEG signals, classification of seizures, classification of delirium, classification of stroke, classification of sedation, generate notifications, generate probability plots of neurological conditions, processing EEG signals, segmenting EEG signals, extracting features, processing features with machine learning algorithms, relating features to assessment scales, implementing the control policy and neurological condition burden, calculating the value, plotting the probability of the neurological conditions, etc. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The communication interface may be wired or wireless. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user (e.g., neurological condition detection system manager, neurological condition detection system user, neurological condition data acquirer, neurological condition detection system scribe). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, a login screen for an administrator to access software programmed to control the multi-indication detection system and functionality and/or for providing the operation status health of the multi-indication detection system. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, be component of software described elsewhere herein and may modulate the seizure detection system parameters (e.g., processing EEG signals, machine learning algorithms, control policy, neurological condition burden, notifications, etc.).

### EXAMPLES

### Example 1: Clinical Study Using a Delirium Detection Monitor

A pilot clinical study was performed using the delirium detection monitor illustrated in FIG. 7. The study obtained EEG recordings from 94 total subjects. The first cohort of subjects consisted of 81 ICU patients who underwent EEG recording with the delirium detection monitor as part of their routine clinical care. Out of the 81 ICU patients, 69 were delirium-positive and 12 were delirium-negative. For these patients, delirium assessment was performed using the CAM-ICU by trained clinical nurses experienced in delirium assessment. A second cohort of subjects consisted of 13 healthy volunteers who underwent EEG recording with the delirium detection monitor in a research setting that simulated hospital conditions. All 13 healthy volunteer subjects were delirium-negative. As further detailed in Table 1 below, the 94-subject pilot study dataset was divided into two groups: about ⅔ of the subjects (63 subjects) were used for the delirium detection monitor algorithm development and training, and about ⅓ of the subjects (31 subjects) were used for algorithm validation. The datasets were strictly segregated. There was no crossover of subjects between the development and validation datasets, and no data from the validation dataset was used for algorithm training.

TABLE 1

| | Total | Delirium | Delirium |
|---|---|---|---|
| | Subjects | Positive | Negative |
| Pilot study data set total | 94 | 69 | 25 |
| Development/training | 63 | 50 | 13 |
| dataset | | | |
| Validation dataset | 31 | 19 | 12 |

The performance of the delirium detection monitor was evaluated by calculating the sensitivity and specificity with respect to correct identification of delirium-positive patients. The performance from the validation dataset is shown below in Table 2. As shown in the table, use of the delirium detection monitor achieved clinical success, resulting in a sensitivity of about 95% and specificity of about 92% in the detection of delirium.

TABLE 2

| | Validation Dataset | 95% Confidence |
|---|---|---|
| | Performance | Interval |
| Sensitivity | 94.7% | 79%-100% |
| Specificity | 91.7% | 67%-100% |

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated.

1. A method for detecting delirium comprising:
obtaining data comprising a plurality of electroencephalography (EEG) signals recorded over a plurality of channels from a subject;
pre-processing the data by:
dividing the EEG signal into a plurality of temporal segments, each temporal segment corresponding to a time epoch defined by at least a start time and a duration; and
extracting a plurality of features from each of the plurality of temporal segments;
using one or more machine learning models to generate a delirium classification for each of the temporal segments based on the extracted plurality of features; and
determining an overall delirium score for the subject during a time-window, the overall delirium score being based on the delirium classifications generated by the one or more machine learning models, and the time window comprising one or more time epochs.

2. The method of claim 1, wherein the delirium classification is a binary classification that is delirium-positive or delirium-negative, a delirium probability value, or a delirium severity value.

3. The method of claim 1, further comprising providing a trace of the overall delirium score over time.

4. The method claim 3, further comprising determining a trendline of the trace.

5. The method of claim 1, wherein pre-processing the data further comprises extracting a plurality of multi-channel features that quantify a degree of correlation between pairs of temporal segments from different EEG signals corresponding to a given time epoch.

6. The method of claim 5, further comprising using a multichannel machine learning model to generate a multi-channel delirium classification for each time epoch based on the plurality of multi-channel features, and wherein the delirium score is further based on the multi-channel delirium classification.

7. The method of claim 1, wherein the delirium is hypo-active delirium.

8. The method of claim 1, wherein the time-window has a duration that encompasses one time epoch.

9. The method of claim 1, wherein the time-window has a duration that encompasses a plurality of successive time epochs.

10. The method of claim 1, wherein the duration of each of the time epochs ranges from about 1 second to about 10 minutes.

11. The method of claim 10, wherein the duration of each of the time epochs is about 10 seconds, about 30 seconds, about 60 seconds, about 2 minutes, about 5 minutes, or about 10 minutes.

12. The method of claim 1, wherein successive time epochs are non-overlapping.

13. The method of claim 1, wherein successive time epochs overlap by 50% or less.

14. The method of claim 1, wherein the plurality of features comprises at least one time-domain feature.

15. The method of claim 1, wherein the plurality of features comprises at least one frequency-domain feature.

16. The method of claim 1, wherein the plurality of features comprises at least one feature that quantifies a degree of correlation of at least one of the plurality of temporal segments with a corresponding time-based segment of at least one other simultaneously collected EEG signal.

17. The method of claim 16, wherein the EEG signal from the at least one of the plurality of temporal segments and the at least one other simultaneously collected EEG signal is collected from the same hemisphere of a brain.

18. The method of claim 1, wherein each channel is assigned to an independent machine learning model, and wherein for each channel, the extracted features are applied to the machine learning model corresponding to the channel.

19. The method of claim 1, wherein the one or more machine learning models is a random forest model.

20. The method of claim 1, wherein the plurality of EEG signals is obtained from a plurality of electrodes incorporated into a headband.

21. The method of claim 1, wherein the plurality of channels comprises 8 channels.

22. The method of claim 1, wherein the plurality of channels comprises 16 channels.

23. The method of claim 1, wherein the detected delirium is hypo-active delirium.

24. The method of claim 1, further comprising treating delirium if delirium is detected.

25. A system for detecting delirium comprising:
a data module configured to receive data comprising a plurality of electroencephalography (EEG) signals recorded during a time window and over a plurality of channels from a subject; and
a delirium detection module comprising a memory storing a set of instructions and one or more processors that are configured to, responsive to the set of instructions:
pre-process the data received by the data module by:
dividing the EEG signal into a plurality of temporal segments, each temporal segment corresponding to a time epoch defined by at least a start time and a duration; and
extracting a plurality of features from each of the plurality of temporal segments;
use one or more machine learning models to generate a delirium classification for each of the temporal segments based on the extracted plurality of features; and
determine an overall delirium score based on the delirium classifications generated by the one or more machine learning models.

26. The system of claim 25, wherein the plurality of channels comprises 8 channels.

27. The system of claim 25, wherein the plurality of channels comprises 16 channels.

28. The system of claim 25, wherein each channel of the plurality of channels is assigned to an independent machine learning model, and wherein for each channel, the extracted plurality of features are applied to the machine learning model corresponding to each channel.

29. The system of claim 25, wherein the one or more machine learning models comprises a random forest model.

30. The system of claim 25, further comprising a headband, the headband comprising a plurality of electrodes from which the plurality of electroencephalography (EEG) signals is recorded.
